(19) United States
(12) Patent Application Publication
(10) Pub. No.: US 2024/0009155 A1
(43) Pub. Date: Jan. 11, 2024

(54) USE OF SHORT CHAIN FATTY ACIDS IN CANCER PREVENTION

(71) Applicant: Temple University-Of The Commonwealth System of Higher Education, Philadelphia, PA (US)

(72) Inventors: Mark A. Feitelson, North Wales, PA (US); Alla Arzumanyan, Huntington Valley, PA (US); Ira Spector, Jenkintown, PA (US)

(21) Appl. No.: 18/252,222

(22) PCT Filed: Nov. 12, 2021
(86) PCT No.: PCT/US21/59211

§ 371 Date: May 9, 2023

Related U.S. Application Data
(60) Provisional application No. 63/112,783, filed on Nov. 12, 2020.

(51) Int. Cl. A61K 31/19; A61P 35/00; A61K 33/08

(52) U.S. Cl. CPC A61K 31/19; A61P 35/00; A61K 33/08

(57) ABSTRACT

The present disclosure describes compositions and methods for treating hepatitis B virus-associated hepatocellular carcinoma. Chronic infection with hepatitis B virus (HBV) is a major risk factor for the development of hepatocellular carcinoma (HCC). The HBV encoded oncoprotein, HBx, alters the expression of host genes and the activity of multiple signal transduction pathways. Short chain fatty acids can be used to delay or block the progression of chronic liver disease to HCC by targeting functions associated with HBx.

CROSS REFERENCE

This application claims the benefit of U.S. Provisional Application No. 63/112,783, filed Nov. 12, 2020, which is incorporated herein by reference.

### BACKGROUND

Chronic infection with hepatitis B virus (HBV) is a major risk factor for the development of hepatocellular carcinoma (HCC). The HBV encoded oncoprotein, HBx, alters the expression of host genes and the activity of multiple signal transduction pathways. Short chain fatty acids (SCFAs) having anti-inflammatory and anti-neoplastic properties can block the progression of chronic liver disease (CLD) to HCC.

### INCORPORATION BY REFERENCE

Each patent, publication, and non-patent literature cited in the application is hereby incorporated by reference in its entirety as if each was incorporated by reference individually.

### SUMMARY OF THE INVENTION

In some embodiments, disclosed herein is a method of treating hepatocellular carcinoma, the method comprising administering to a subject in need thereof of a pharmaceutical composition, the pharmaceutical composition comprising a therapeutically-effective amount of a first compound that is a first short chain fatty acid or a pharmaceutically-acceptable salt thereof, a therapeutically-effective amount of a second compound that is a second short chain fatty acid or a pharmaceutically-acceptable salt thereof, and magnesium hydroxide.

In some embodiments, disclosed herein is a pharmaceutical composition comprising a therapeutically-effective amount of a first compound that is butyric acid or a pharmaceutically-acceptable salt thereof, a therapeutically-effective amount of a second compound that is propionic acid or a pharmaceutically-acceptable salt thereof, and magnesium hydroxide.

In some embodiments, disclosed herein is a pharmaceutical composition consisting essentially of a therapeutically-effective amount of a first compound that is butyric acid or a pharmaceutically-acceptable salt thereof, a therapeutically-effective amount of a second compound that is propionic acid or a pharmaceutically-acceptable salt thereof, and magnesium hydroxide.

### BRIEF DESCRIPTION OF THE DRAWINGS

FIG. 1 shows an outline of experimental steps of a HBx transgenic (HBxTg) mice study described herein.

FIG. 2 Panel A shows the percentage of tumor nodules from the group of 12-month old mice treated with SCFAs or with PBS relative to tumor size. Panel B shows an example of a large tumor from a PBS-treated mouse. Panel C shows an example of two small tumors from a SCFA-treated mouse.

FIG. 3 shows the number of mice with the indicated pathology in livers evaluated at 9 months (Panels A and B) and 12 months (Panels C and D).

FIG. 4 shows the effect of SCFAs on primary human hepatocytes and two HBx expressing human HCC cell lines.

FIG. 5 shows the number of proteins with decreased or increased expression in 12-month old livers from HBxTg mice after SCFA treatment compared to PBS controls arranged by Gene Ontology (GO) biological processes.

FIG. 6 shows immunohistochemical staining for HBx (Panels A and D), Dab2 (Panels B and E), normal IgG (Panel C), or rabbit pre-immune serum (Panel F) in 12-month old mouse livers from animals treated with PBS (Panels A-C) or SCFAs (Panels D-F). Panel G shows a representative Western blot of Dab2 and Shoc2 from treated (T) compared to control (C) livers. Panel H shows a summary of differentially expressed Dab2 and Shoc2 from treated (n=12) compared to control (n=12) mice (*P<0.01). Panel I shows a summary of proteomics data of SCFAs on Ras-related proteins in 12-month old HBxTg mouse livers.

FIG. 7, Panel A shows a pulldown assay for activated Ras in three different mice treated as controls (C) with PBS and three mice treated with the test compounds (T), SCFAs, prior to analysis. Panel B shows a summary of Ras pulldown in seven mice treated with PBS compared to another seven treated with SCFAs (*P<0.001).

FIG. 8 shows a summary of pathway analysis of 12-month old SCFA-treated vs control HBxTg livers. The pathways shown were consistently up-regulated by HBx and down-regulated by SCFAs.

### DETAILED DESCRIPTION

Liver cancer is the sixth most commonly diagnosed and second most lethal cancer worldwide. HCC accounts for about 80% of global primary liver cancer diagnoses. The incidence of HCC continues to increase, with rates tripling in the U.S. over the past twenty years. Chronic infection with HBV is a major risk factor for HCC. HBV has infected roughly 2 billion people worldwide, and among these, an estimated 250 million become carriers who are at increased risk for the development of hepatitis, cirrhosis, and HCC. In the U.S., the two-year survival rate from the time of diagnosis is less than 50%, and 5 year survival rate is only 8.9%, thereby highlighting the urgent need for more effective therapeutic options.

Chronic infection with hepatitis B virus (HBV) is a major risk factor for the development of hepatocellular carcinoma (HCC). The HBV-encoded oncoprotein, HBx, alters the expression of host genes and the activity of multiple signal transduction pathways. HBV encoded HBx can contribute centrally to the development of HCC.

Recurrent cycles of cell death and regeneration in CLD are associated with increased integration of the HBx gene into host DNA, and production of functional HBx, thereby leading to alterations of host gene expression, chromosomal instability, and altered signaling pathways crucial to cell survival, inflammation, angiogenesis, and immune responses. Among the signaling pathways altered by HBx, the PI3K, Ras, and NF-κB signaling pathways promote cell survival and growth. Aberrant activation of PI3K, PDGF, VEGF, Ras, and NF-κB by HBx can lead to HCC initiation and progression. HBx can also alter host gene expression through epigenetic regulation, by stimulating histone deacetylases (HDACs) and DNA methyltransferases (DNMTs), HBx is capable of silencing tumor suppressors and activating host oncogenes to promote carcinogenesis.

HCC change can alter the composition of the gut microbiome. This change can correspond to changes in the levels and ratios of pro-inflammatory and anti-inflammatory metabolites in the gut. SCFAs are produced by the anaerobic fermentation of dietary fiber carried out by gut microorganisms. SCFAs regulate cell growth and differentiation, prevent or lessen the likelihood of inflammation, inhibit cell proliferation, and induce apoptosis in cancer cells. SCFAs can oppose the actions of HBx on many of the same molecules and pathways that HBx exploits in carcinogenesis. For example, SCFAs can reduce cell proliferation by inhibiting histone deacetylases (HDACi), whereas HBx stimulates the activity of selected HDACs. Thus, SCFAs can reverse the epigenetic effects of HBx on chromatin structure. SCFAs inhibit pro-inflammatory NF-κB signaling, while HBx stimulates NFκKB and associated inflammation. Specifically, HBx expression and activity are increased in an oxidative environment characteristic of inflammation. Further, the intensity and distribution of intrahepatic HBx expression correlates with the severity of CLD, thereby suggesting that a chronic inflammatory environment potentiates the actions of HBx.

Short chain fatty acids (SCFAs) having anti-inflammatory and anti-neoplastic properties can be used to block the progression of chronic liver disease (CLD) to HCC. Disclosed herein are methods of blocking the progression of CLD to HCC using pharmaceutical compositions comprising SCFAs.

Disclosed herein are methods of treating hepatocellular carcinoma, the method comprising administering to a subject in need thereof of a pharmaceutical composition, the pharmaceutical composition comprising a therapeutically-effective amount of a first compound that is a first short chain fatty acid or a pharmaceutically-acceptable salt thereof, a therapeutically-effective amount of a second compound that is a second short chain fatty acid or a pharmaceutically-acceptable salt thereof, and magnesium hydroxide. Further disclosed herein is a pharmaceutical composition comprising a therapeutically-effective amount of a first compound that is butyric acid or a pharmaceutically-acceptable salt thereof, a therapeutically-effective amount of a second compound that is propionic acid or a pharmaceutically-acceptable salt thereof, and magnesium hydroxide. Also disclosed herein is a pharmaceutical composition consisting essentially of a therapeutically-effective amount of a first compound that is butyric acid or a pharmaceutically-acceptable salt thereof, a therapeutically-effective amount of a second compound that is propionic acid or a pharmaceutically-acceptable salt thereof, and magnesium hydroxide.

### Compounds of the Disclosure

SCFAs are saturated aliphatic acids consisting of one polar carboxylic acid moiety and a hydrophobic hydrocarbon chain. The present disclosure describes use of a SCFA, a SCFA precursor, a SCFA biosynthesis precursor, a compound comprising a SCFA moiety, a SCFA derivative, or a pharmaceutically-acceptable salt thereof.

In some embodiments, a compound of the disclosure is formic acid, acetic acid, propionic acid, butyric acid, isobutyric acid, valeric acid, or isovaleric acid. In some embodiments, a compound of the disclosure is formate, acetate, propionate, butyrate, isobutyrate, valerate, or isovalerate, or a pharmaceutically-acceptable salt thereof. In some embodiments, a compound of the disclosure is sodium formate, sodium acetate, sodium propionate, sodium butyrate, sodium isobutyrate, sodium valerate, or sodium isovalerate. In some embodiments, a compound of the disclosure is methoxyacetic acid, valproic acid, 3-methoxypropionic acid, ethoxyacetic acid, tributyrin, or propionate ester. In some embodiments, a compound of the disclosure is butyrate, N-acetylbutyrate, phenylbutyrate, isobutyrate, pivaloyloxymethylbutyrate, or monoacetone glucose-3-butyrate. In some embodiments, a compound of the disclosure is sodium butyrate, sodium N-acetylbutyrate, sodium phenylbutyrate, sodium isobutyrate, sodium pivaloyloxymethylbutyrate, or sodium monoacetone glucose-3-butyrate.

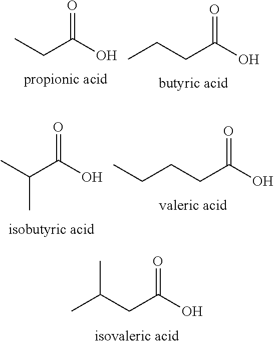

In some embodiments, a compound of the disclosure is pyruvic acid, octanoic acid, dodecanoic acid, (4R)-4-hydroxypentanoic acid, 2-ethylhydracrylic acid, 2-hydroxy-3-methylpentanoic acid, 2-methylbut-2-enoic acid, butanoic acid, methylbutyric acid, dimethylbutyric acid, pentadienoic acid, pentenoic acid, pivalic acid, or propynoic acid. In some embodiments, a compound of the disclosure is pyruvate, octanoate, dodecanoate, (4R)-4-hydroxypentanoate, 2-ethylhydracrylate, 2-hydroxy-3-methylpentanoate, 2-methylbut-2-enoate, butanoate, methylbutyrate, dimethylbutyrate, pentadienoate, pentenoate, pivalate, or propynoate, or a pharmaceutically-acceptable salt of any of the foregoing.

In some embodiments, a compound of the disclosure is a SCFA precursor or derivative thereof. In some embodiments, the compound of the disclosure is lactate, succinate, formate, 1,2-propenediol, tryptamine, indole, indole-3-acetate, or a pharmaceutically-acceptable salt thereof.

In some embodiments, a compound of the disclosure is butyric acid or a pharmaceutically-acceptable salt thereof. In some embodiments, a compound of the disclosure is sodium butyrate. In some embodiments, a compound of the disclosure is propionic acid or a pharmaceutically-acceptable salt thereof. In some embodiments, a compound of the disclosure is sodium propionate.

In some embodiments, a compound of the disclosure is a SCFA biosynthesis precursor or derivative thereof. In some embodiments, a compound of the disclosure is an acetyl-CoA carboxylase inhibitor, an adenosine monophosphate kinase (AMPK) activator, or vitamin D.

### Pharmaceutically Acceptable Salts

The disclosure provides pharmaceutically-acceptable salts of any therapeutic compound described herein. In some embodiments, the disclosure provides pharmaceutically-acceptable hydrates or solvates of compounds described herein. In some embodiments, the disclosure provides base-addition salts. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the disclosure. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

In some embodiments, a compound of the disclosure is an ester of the carboxylic acid. In some embodiments, a compound of the disclosure is an ester of the carboxylic acid with a branched or unbranched alkyl alcohol of 1 to 6 carbon atoms. In some embodiments, a compound of the disclosure can be an ethyl ester, propyl ester, butyl ester, isopropyl ester, t-butyl ester, pentyl ester, or hexyl ester.

In some embodiments, pharmaceutical compositions of the disclosure comprise butyric acid or a pharmaceutically-acceptable salt thereof. In some embodiments, pharmaceutical compositions of the disclosure comprise sodium butyrate. In some embodiments, pharmaceutical compositions of the disclosure comprise propionic acid or a pharmaceutically-acceptable salt thereof. In some embodiments, pharmaceutical compositions of the disclosure comprise sodium propionate.

### Pharmaceutical Compositions of the Disclosure

The present disclosure provides pharmaceutical compositions comprising at least one compound of the disclosure. A pharmaceutical composition of the disclosure can be a combination of any compounds described herein with other chemical components, such as carriers, stabilizers, diluents, dispersing agents, suspending agents, thickening agents, and/or excipients. The pharmaceutical composition facilitates administration of the compound to an organism, for example, a subject. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration.

A pharmaceutical composition can be administered in a local manner, for example, via injection of the compound directly into an organ, optionally in a depot or sustained release formulation or implant.

In some embodiments, a pharmaceutical composition of the disclosure is formulated for oral administration. In some embodiments, a pharmaceutical composition can be formulated by combining compounds of the disclosure with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

In some embodiments, pharmaceutical preparations of the disclosure can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents.

Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives. The compounds of the disclosure can be applied topically to the skin, or a body cavity, for example, oral, vaginal, bladder, cranial, spinal, thoracic, or pelvic cavity of a subject. The compounds of the disclosure can be applied to an accessible body cavity.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the disclosure include liquid, powder, gel, nanosuspension, nanoparticle, microgel, aqueous or oily suspensions, emulsion, and any combination thereof.

Non-limiting examples of pharmaceutically-acceptable excipients suitable for use in the disclosure include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof. In some embodiments, the pharmaceutically-acceptable excipients of the disclosure are pharmaceutical grade excipients.

Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release. A pharmaceutical composition of the disclosure can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some embodiments, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16, or about 24 hours.

An enteric coating is a polymer barrier applied on oral medication that prevents dissolution or disintegration in the gastric environment. Enteric coatings can protect drugs from the acidity of the stomach, the stomach from detrimental effects of the drug, or to release the drug after the stomach. In some embodiments, the pharmaceutical compositions of the disclosure are provided with an enteric coating. In some embodiments, the enteric coatings of the disclosure are pharmaceutical grade enteric coatings. In some embodiments, the pharmaceutical compositions of the disclosure are provided with an enteric coating that dissolves in the lower gastrointestinal track.

In some embodiments, a material used to provide an enteric coating to a compound of the disclosure is a fatty acid, wax, shellac, plastic, plant fiber, or a film resin. In some embodiments, the enteric coating material is methyl acrylate-methacrylate acid copolymers, cellulose acetate phthalate (CAP), cellulose acetate succinate, hydroxypropyl methyl cellulose phthalate, hydroxypropyl methyl cellulose acetate succinate, hypromellose acetate succinate, polyvinyl acetate phthalate (PVAP), methyl methacrylate-methacrylic acid copolymers, shellac, cellulose acetate trimellitate, sodium alginate, or zein. In some embodiments, the compound is provided as an enteric-coated soft gel, wherein the enteric coating is provided as an enteric coating aqueous solution. In some embodiments, the enteric coating aqueous solution is ethylcellulose, medium chain triglycerides, oleic acid, sodium alginate, or stearic acid.

In some embodiments, the enteric coating is provided as a Vcaps® enteric capsule. In some embodiments, the enteric coating is cellulose acetate phthalate, cellulose acetate trimellitate (CAT), polyvinyl acetate phthalate, hydroxypropylmethylcellulose phthalate, hydroxypropylmethylcellulose acetate succinate, poly (1:1 methacrylic acid:ethyl acrylate), poly (1:1 methacrylic acid:methyl methacrylate), or poly (1:2 methyacrylic acid:methyl methacrylate). In some embodiments, the enteric coating is Eudragit® L30D, Eudragit® L100-55, HP-F, Sureteric®, Acryl-Eze®, Aquarius™ Control ENA, Aquateric™, Aquacoat® ECD, or Aquasolve™.

The enteric coating used to coat a pharmaceutical composition of the disclosure can have a thickness of from about 0.5 μm to about 500 μm. In some embodiments, the enteric coating can have a thickness of from about 0.5 μm to about 5 μm, about 5 μm to about 20 μm, about 20 μm to about 50 μm, about 50 μm to about 100 μm, about 100 μm to about 200 μm, about 200 μm to about 300 μm, about 300 μm to about 400 μm, or about 400 μm to about 500 μm. In some embodiments, the enteric coating can have a thickness of about 0.5 μm, about 10 μm, about 25 μm, about 50 μm, about 75 μm, about 100 μm, about 150 μm, about 200 μm, about 250 μm, about 300 μm, about 350 μm, about 400 μm, about 450 μm, or about 500 μm. In some embodiments, the enteric coating has a thickness of about 200 μm. In some embodiments, the enteric coating has a thickness of about 350 μm. In some embodiments, the enteric coating has a thickness of about 500 μm.

Depending on the intended mode of administration, the pharmaceutical compositions can be in the form of solid, semi-solid or liquid dosage forms, such as, for example, tablets, suppositories, pills, capsules, powders, liquids, elixir, suspensions, lotions, creams, or gels, for example, in unit dosage form suitable for single administration of a precise dosage. In some embodiments, a pharmaceutical composition can be in the form of a nanosuspension, aqueous suspension, or oily suspension. In some embodiments, a pharmaceutical composition of the disclosure can be in the form of a drop or syrup.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof. In some embodiments, pharmaceutically-acceptable excipients suitable for use in the disclosure also include adjuvants, anti-oxidants, chelating agents, surfactants, foaming agents, wetting agents, emulsifying agents, viscosifiers, buffering agents, or preservatives.

In some embodiments, the pharmaceutically-acceptable excipient is a permeation enhancer. In some embodiments, the permeation enhancer is ethanol, glycerol monolaurage, polyethylene glycol monolaurate, or dimethylsulfoxide. In some embodiments, the permeation enhancer is oleic acid, oleyl alcohol, ethoxydiglycol, laurocapram, alkanecarboxylic acids, dimethylsulfoxide, polar lipids, or N-methyl-2-pyrrolidone.

In some embodiments, the pharmaceutically-acceptable excipient is a hydrotropic agent. In some embodiments, the hydrotropic agent is isopropyl alcohol, propylene glycol, or sodium xylene sulfonate.

In some embodiments, the pharmaceutically-acceptable excipient is a tablet binder, tablet disintegrant, viscosity increasing agent, tablet or capsule diluent, tablet or capsule disintegrant, thermal stabilizer, adsorbent, film-forming agent, granulating agent, coating agent, flavoring fixative, coloring agent, sweetening agent, or tonicity agent.

In some embodiments, the pharmaceutically-acceptable excipient is acacia, alginate, alginic acid, aluminum acetate, benzyl alcohol, butyl paraben, butylated hydroxy toluene, citric acid, calcium carbonate, candelilla wax, croscarmellose sodium, confectioner sugar, colloidal silicone dioxide, cellulose, plain or anhydrous calcium phosphate, carnauba wax, corn starch, caboxymethylcellulose calcium, calcium stearate, calcium disodium EDTA, copolyvidone, hydrogenated castor oil, calcium hydrogen phosphate dihydrate, cetylpyridine chloride, cysteine HCl, crosspovidone, calcium phosphate di or tri basic, dibasic calcium phosphate, disodium hydrogen phosphate, dimethyicone, erythrosine sodium, ethyl cellulose, gelatin, glyceryl monooleate, glycerine, glycine, glyceryl monostearate, glyceryl behenate, hydroxypropyl cellulose, hydroxy propyl methyl cellulose, hypromellose, HPMC phthalate, iron oxides, iron oxide yellow, iron oxide red, lactose (hydrous or anhydrous), magnesium stearate, microcrystalline cellulose, mannitol, methyl cellulose, magnesium carbonate, mineral oil, methacrylic acid copolymer, magnesium oxide, methyl paraben, povidone (PVP), polyethylene glycol (PEG), polysorbate 80, propylene glycol, polyethylene oxide, propylene paraben, polaxamer (407, 188, or plain), potassium bicarbonate, potassium sorbate, potato starch, phosphoric acid, polyoxy140 stearate, sodium starch glycolate, pregelatinized starch, sodium crossmellose, sodium lauryl sulfate, starch, silicone dioxide, sodium benzoate, stearic acid, sucrose, sorbic acid, sodium carbonate, saccharin sodium, sodium alginate, silica gel, sorbiton monooleate, sodium stearyl fumarate, sodium chloride, sodium metabisulfite, sodium citrate dihydrate, sodium starch, sodium carboxymethyl cellulose, succinic acid, sodium propionate, titanium dioxide, talc, triacetin, or triethyl citrate.

In some embodiments, a pharmaceutical composition of the disclosure can comprise a stabilizer. In some embodiments, a pharmaceutical composition of the disclosure comprises magnesium hydroxide. In some embodiments, a pharmaceutical composition of the disclosure comprises at most about 3.0%, at most about 2.8%, at most about 2.6%, at most about 2.4%, at most about 2.2%, at most about 2.0%, at most about 1.8%, at most about 1.6%, at most about 1.4%, at most about 1.2%, at most about 1.0%, at most about 0.9%, at most about 0.8%, at most about 0.7%, at most about 0.6%, at most about 0.5%, at most about 0.4%, at most about 0.3%, at most about 0.2%, or at most about 0.1% (w/w) of magnesium hydroxide. In some embodiments, a pharmaceutical composition of the disclosure comprises at most about 3.0% (w/w) of magnesium hydroxide. In some embodiments, a pharmaceutical composition of the disclosure comprises at most about 2.0% (w/w) of magnesium hydroxide. In some embodiments, a pharmaceutical composition of the disclosure comprises at most about 1.0% (w/w) of magnesium hydroxide. In some embodiments, a pharmaceutical composition of the disclosure comprises at most about 0.5% (w/w) of magnesium hydroxide. In some embodiments, a pharmaceutical composition of the disclosure comprises at most about 0.3% (w/w) of magnesium hydroxide. In some embodiments, a pharmaceutical composition of the disclosure comprises at most about 0.1% (w/w) of magnesium hydroxide.

In some embodiments, a pharmaceutical composition of the disclosure comprises butyric acid or a pharmaceutically-acceptable salt thereof, propionic acid or a pharmaceutically-acceptable salt thereof, and magnesium hydroxide. In some embodiments, a pharmaceutical composition of the disclosure consists essentially of butyric acid or a pharmaceutically-acceptable salt thereof, propionic acid or a pharmaceutically-acceptable salt thereof, and magnesium hydroxide.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

In practicing the methods of treatment or use provided herein, therapeutically-effective amounts of the compounds described herein are administered in pharmaceutical compositions to a subject having a disease or condition to be treated. In some embodiments, the subject is a mammal such as a human. In some embodiments, the subject is an adult, elderly adult, adolescent, pre-adolescent, child, toddler, infant, neonate, or a non-human children, toddlers, infants, neonates, and non-human animals. In some embodiments, a subject is a patient.

Non-limiting examples of pharmaceutically active agents suitable for combination with compositions of the disclosure include anti-infectives, i.e., aminoglycosides, antiviral agents, antimicrobials, anticholinergics/antispasmotics, antidiabetic agents, antihypertensive agents, antineoplastics, cardiovascular agents, central nervous system agents, coagulation modifiers, hormones, immunologic agents, immunosuppressive agents, and ophthalmic preparations.

In some embodiments, a pharmaceutical composition of the disclosure comprises: a) a first short chain fatty acid or a pharmaceutically-acceptable salt thereof; and b) a second short chain fatty acid or a pharmaceutically-acceptable salt thereof, wherein the first short chain fatty acid or the pharmaceutically-acceptable salt thereof and the second short chain fatty acid or the pharmaceutically-acceptable salt thereof are present in the formulation in a ratio of at least 2:1, at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, at least 16:1, at least 17:1, at least 18:1, at least 19:1, or at least 20:1. In some embodiments, the first short chain fatty acid or the pharmaceutically-acceptable salt thereof and the second short chain fatty acid or the pharmaceutically-acceptable salt thereof are present in the formulation in a ratio of at least 4:1. In some embodiments, the first short chain fatty acid or the pharmaceutically-acceptable salt thereof and the second short chain fatty acid or the pharmaceutically-acceptable salt thereof are present in the formulation in a ratio of at least 10:1. In some embodiments, the first short chain fatty acid or the pharmaceutically-acceptable salt thereof and the second short chain fatty acid or the pharmaceutically-acceptable salt thereof are present in the formulation in a ratio of at least 15:1.

In some embodiments, a pharmaceutical composition of the disclosure comprises: a) a first short chain fatty acid or a pharmaceutically-acceptable salt thereof; b) a second short chain fatty acid or a pharmaceutically-acceptable salt thereof; and magnesium hydroxide, wherein the first short chain fatty acid or the pharmaceutically-acceptable salt thereof and the second short chain fatty acid or the pharmaceutically-acceptable salt thereof are present in the formulation in a ratio of from about 2:1 to about 4:1, from about 4:1 to about 6:1, from about 6:1 to about 8:1, from about 8:1 to about 10:1, from about 10:1 to about 12:1, from about 12:1 to about 14:1, from about 14:1 to about 16:1, from about 16:1 to about 18:1, or from about 18:1 to about 20:1. In some embodiments, the first short chain fatty acid or the pharmaceutically-acceptable salt thereof and the second short chain fatty acid or the pharmaceutically-acceptable salt thereof are present in the formulation in a ratio of from about 14:1 to about 16:1.

In some embodiments, a pharmaceutical composition of the disclosure comprises: a) a first short chain fatty acid or a pharmaceutically-acceptable salt thereof; b) a second short chain fatty acid or a pharmaceutically-acceptable salt thereof; and magnesium hydroxide, wherein the first short chain fatty acid or the pharmaceutically-acceptable salt thereof and the second short chain fatty acid or the pharmaceutically-acceptable salt thereof are present in the formulation in a ratio of about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1. In some embodiments, the first short chain fatty acid or the pharmaceutically-acceptable salt thereof and the second short chain fatty acid or the pharmaceutically-acceptable salt thereof are present in the formulation in a ratio of about 10:1. In some embodiments, the first short chain fatty acid or the pharmaceutically-acceptable salt thereof and the second short chain fatty acid or the pharmaceutically-acceptable salt thereof are present in the formulation in a ratio of about 15:1.

In some embodiments, the first short chain fatty acid or the pharmaceutically-acceptable salt thereof is butyric acid or a pharmaceutically-acceptable salt thereof. In some embodiments, the first short chain fatty acid or the pharmaceutically-acceptable salt thereof is sodium butyrate. In some embodiments, the first short chain fatty acid is butyric acid. In some embodiments, the second short chain fatty acid or the pharmaceutically-acceptable salt thereof is propionic acid or a pharmaceutically-acceptable salt thereof. In some embodiments, the second short chain fatty acid is sodium propionate. In some embodiments, the second short chain fatty acid is propionic acid. In some embodiments, the pharmaceutical composition further comprises a pharmaceutically-acceptable excipient. In some embodiments, the pharmaceutically-acceptable excipient is cellulose. In some embodiments, the pharmaceutically-acceptable excipient is methylcellulose. In some embodiments, the pharmaceutically-acceptable excipient is hydroxypropyl cellulose. In some embodiments, the pharmaceutical composition further comprises an enteric coating. In some embodiments, the enteric coating is a Vcaps® (hydroxypropyl methylcellulose) enteric capsule. In some embodiments, the enteric coating is CAT. In some embodiments, the pharmaceutical composition is formulated as a tablet. In some embodiments, the pharmaceutical composition is formulated as a capsule.

In some embodiments, a pharmaceutical composition of the disclosure comprises: a) butyric acid or a pharmaceutically-acceptable salt thereof; b) propionic acid or a pharmaceutically-acceptable salt thereof; and magnesium hydroxide, wherein butyric acid or a pharmaceutically-acceptable salt thereof and propionic acid or a pharmaceutically-acceptable salt thereof are present in the pharmaceutical composition in a ratio of at least 4:1, at least 5:1, at least 6:1, at least 7:1, at least 8:1, at least 9:1, at least 10:1, at least 11:1, at least 12:1, at least 13:1, at least 14:1, at least 15:1, at least 16:1, at least 17:1, at least 18:1, at least 19:1, or at least 20:1. In some embodiments, a pharmaceutical composition of the disclosure comprises: a) sodium butyrate; b) sodium propionate; and magnesium hydroxide, wherein the sodium butyrate and sodium propionate are present in the pharmaceutical composition in a ratio of at least 4:1, at least 5:1, at least 6:1, at least 7:1, at least 8:1, at least 9:1, at least 10:1, at least 11:1, at least 12:1, at least 13:1, at least 14:1, at least 15:1, at least 16:1, at least 17:1, at least 18:1, at least 19:1, or at least 20:1. In some embodiments, the first short chain fatty acid or the pharmaceutically-acceptable salt thereof and the second short chain fatty acid or the pharmaceutically-acceptable salt thereof are present in the formulation in a ratio of at least 4:1. In some embodiments, the first short chain fatty acid or the pharmaceutically-acceptable salt thereof and the second short chain fatty acid or the pharmaceutically-acceptable salt thereof are present in the formulation in a ratio of at least 10:1. In some embodiments, the first short chain fatty acid or the pharmaceutically-acceptable salt thereof and the second short chain fatty acid or the pharmaceutically-acceptable salt thereof are present in the formulation in a ratio of at least 15:1.

In some embodiments, a pharmaceutical composition of the disclosure comprises: a) butyric acid or a pharmaceutically-acceptable salt thereof; b) propionic acid or a pharmaceutically-acceptable salt thereof; and magnesium hydroxide, wherein butyric acid or a pharmaceutically-acceptable salt thereof and propionic acid or a pharmaceutically-acceptable salt thereof are present in the pharmaceutical composition in a ratio of about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1. In some embodiments, a pharmaceutical composition of the disclosure comprises: sodium butyrate; sodium propionate; and magnesium hydroxide, wherein the sodium butyrate and sodium propionate are present in the pharmaceutical composition in a ratio of about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1. In some embodiments, the first short chain fatty acid or the pharmaceutically-acceptable salt thereof and the second short chain fatty acid or the pharmaceutically-acceptable salt thereof are present in the formulation in a ratio of about 4:1. In some embodiments, the first short chain fatty acid or the pharmaceutically-acceptable salt thereof and the second short chain fatty acid or the pharmaceutically-acceptable salt thereof are present in the formulation in a ratio of about 10:1. In some embodiments, the first short chain fatty acid or the pharmaceutically-acceptable salt thereof and the second short chain fatty acid or the pharmaceutically-acceptable salt thereof are present in the formulation in a ratio of about 15:1.

In some embodiments, the first short chain fatty acid is butyric acid or a pharmaceutically-acceptable salt thereof. In some embodiments, the first short chain fatty acid is butyric acid or a pharmaceutically-acceptable salt thereof. In some embodiments, the first short chain fatty acid is sodium butyrate. In some embodiments, the second short chain fatty acid is propionic acid or a pharmaceutically-acceptable salt thereof. In some embodiments, the second short chain fatty acid is propionic acid. In some embodiments, the second short chain fatty acid is sodium propionate. In some embodiments, the pharmaceutical composition further comprises a pharmaceutically-acceptable excipient. In some embodiments, the pharmaceutically-acceptable excipient is cellulose. In some embodiments, the pharmaceutically-acceptable excipient is methylcellulose. In some embodiments, the pharmaceutically-acceptable excipient is hydroxypropyl cellulose. In some embodiments, the pharmaceutical composition further comprises an enteric coating. In some embodiments, the enteric coating is a Vcaps® (hydroxypropyl methylcellulose) enteric capsule. In some embodiments, the enteric coating is CAT. In some embodiments, the pharmaceutical composition is formulated as a tablet. In some embodiments, the pharmaceutical composition is formulated as a capsule.

### Dosing

Pharmaceutical compositions described herein can be in unit dosage forms suitable for single administration of precise dosages. In unit dosage form, the formulation is divided into unit doses containing appropriate quantities of one or more pharmaceutical compositions or formulations. The unit dosage can be in the form of a package containing discrete quantities of the pharmaceutical composition or formulation.

In some embodiments, a pharmaceutical composition or formulation of the disclosure is provided as a liquid in a vial or ampoule. In some embodiments, a pharmaceutical composition or formulation of the disclosure is provided as an aqueous suspension packaged in a single-dose non-reclosable container. In some embodiments, a pharmaceutical composition or formulation of the disclosure is provided as an aqueous suspension packaged in a multi-dose reclosable container. Multiple-dose reclosable containers can be used, for example, in combination with a preservative.

In some embodiments, a pharmaceutical composition or formulation of the disclosure is provided as a powder in a single-dose container, for example, a sachet. In some embodiments, a pharmaceutical composition or formulation of the disclosure is provided as a powder in a multi-dose reclosable container. In some embodiments, a pharmaceutical composition or formulation of the disclosure is provided in the form of a tablet. In some embodiments, a pharmaceutical composition or formulation of the disclosure is provided in the form of a capsule.

In some embodiments, a compound described herein can be present in a composition in range of from about 50 mg to about 100 mg, about 100 mg to about 200 mg, about 200 mg to about 300 mg, about 300 mg to about 400 mg, or about 400 mg to about 500 mg. In some embodiments, a compound described herein can be present in a composition in a range of from about 500 mg to about 5,000 mg, from about 1,000 mg to about 5,000 mg, from about 1,500 mg to about 4,000 mg, from about 2,000 mg to about 3,000 mg, or from about 2,500 mg to about 3,000 mg. In some embodiments, a compound described herein can be present in a composition in a range of from about 1,000 mg to about 1,200 mg, from about 1,200 mg to about 1,400 mg, from about 1,400 mg to about 1,600 mg, from about 1,600 mg to about 1,800 mg, from about 1,800 mg to about 2,000 mg, from about 2,000 mg to about 2,200 mg, from about 2,200 mg to about 2,400 mg, from about 2,400 mg to about 2,600 mg, from about 2,600 mg to about 2,800 mg, from about 2,800 mg to about 3,000 mg, from about 3,000 mg to about 3,200 mg, from about 3,200 mg to about 3,400 mg, from about 3,400 mg to about 3,600 mg, from about 3,600 mg to about 3,800 mg, from about 3,800 mg to about 4,000 mg, from about 4,000 mg to about 4,200 mg, from about 4,200 mg to about 4,400 mg, from about 4,400 mg to about 4,600 mg, from about 4,600 mg to about 4,800 mg, or from about 4,800 mg to about 5,000 mg.

In some embodiments, a compound described herein can be present in a composition in an amount of about 50 mg, about 100 mg, about 150 mg, about 200 mg, about 250 mg, about 300 mg, about 350 mg, about 400 mg, about 450 mg, or about 500 mg. In some embodiments, a compound described herein can be present in a composition in an amount of about 500 mg, about 750 mg, about 1,000 mg, about 1,250 mg, about 1,500 mg, about 1,750 mg, about 2,000 mg, about 2,250 mg, about 2,500 mg, about 3,000 mg, about 3,250 mg, about 3,500 mg, about 3,750 mg, about 4,000 mg, about 4,250 mg, about 4,500 mg, about 4,750 mg, or about 5,000 mg.

In some embodiments, a dose can be expressed in terms of an amount of the drug divided by the mass of the subject, for example, milligrams of drug per kilograms of subject body mass. In some embodiments, a compound is administered in an amount ranging from about 1 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, about 25 mg/kg to about 30 mg/kg, about 30 mg/kg to about 35 mg/kg, about 35 mg/kg to about 40 mg/kg, about 40 mg/kg to about 45 mg/kg, or about 45 mg/kg to about 50 mg/kg, about 50 mg/kg to about 55 mg/kg, about 55 mg/kg to about 60 mg/kg, about 60 mg/kg to about 65 mg/kg, about 65 mg/kg to about 70 mg/kg, or about 70 mg/kg to about 75 mg/kg. In some embodiments, a compound is administered in an amount of about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, or about 75 mg/kg.

In some embodiments, a compound is administered in amount of about 1 mg/kg, about 2 mg/kg, about 3 mg/kg, about 4 mg/kg, about 5 mg/kg, or about 6 mg/kg. In some embodiments, a compound is administered in an amount of about 4 mg/kg. In some embodiments, a compound is administered in an amount of about 5 mg/kg.

In some embodiments, a compound is administered in an amount of about 50 mg/kg, about 55 mg/kg, about 60 mg/kg, about 65 mg/kg, or about 70 mg/kg. In some embodiments, a compound is administered in an amount of about 65 mg/kg. In some embodiments, a compound is administered in an amount of about 70 mg/kg.

In some embodiments, a pharmaceutical composition comprises 1, 2, 3, 4, or 5 compounds of the disclosure. In some embodiments, a pharmaceutical composition comprises 1 compound of the disclosure. In some embodiments, a pharmaceutical composition comprises 2 compounds of the disclosure. In some embodiments, a pharmaceutical composition comprises 3 compounds of the disclosure.

In some embodiments, a pharmaceutical composition comprises a first compound of the disclosure and a second compound of the disclosure. In some embodiments, a pharmaceutical composition comprises a first compound in an amount of from about 50 mg to about 4000 mg; and a second compound in an amount of from about 50 mg to about 500 mg. In some embodiments, a pharmaceutical composition comprises a first compound in an amount of about 1500 mg to about 2000 mg; and a second compound in an amount of about 150 mg to about 200 mg. In some embodiments, a pharmaceutical composition comprises a first compound in an amount of about 3000 mg to about 4000 mg; and a second compound in an amount of about 200 mg to about 300 mg. In some embodiments, a pharmaceutical composition comprises a first compound in an amount of from about 2000 mg to about 3000 mg; and a second compound in an amount of from about 300 mg to about 400 mg.

In some embodiments, a pharmaceutical composition comprises a first compound in an amount of about 65 mg/kg to about 70 mg/kg; and a second compound in an amount of about 1 mg/kg to about 5 mg/kg. In some embodiments, a pharmaceutical composition comprises a first compound in an amount of about 65 mg/kg and a second compound in an amount of about 4 mg/kg. In some embodiments, a pharmaceutical composition comprises a first compound in an amount of about 70 mg/kg and a second compound in an amount of 5 mg/kg.

In some embodiments, a pharmaceutical composition comprises butyric acid or a pharmaceutically-acceptable salt thereof and one additional SCFA. In some embodiments, a pharmaceutical composition comprises propionic acid or a pharmaceutically-acceptable salt thereof and butyric acid or a pharmaceutically-acceptable salt thereof. In some embodiments, a pharmaceutical composition comprises isobutyric acid or a pharmaceutically-acceptable salt thereof and butyric acid and a pharmaceutically-acceptable salt thereof. In some embodiments, a pharmaceutical composition comprises butyric acid or a pharmaceutically-acceptable salt thereof and valerate or a pharmaceutically-acceptable salt thereof. In some embodiments, a pharmaceutical composition comprises butyric acid or a pharmaceutically-acceptable salt thereof and isovalerate or a pharmaceutically-acceptable salt thereof.

In some embodiments, a pharmaceutical composition comprises sodium butyrate and one additional SCFA. In some embodiments, a pharmaceutical composition comprises sodium propionate and sodium butyrate. In some embodiments, a pharmaceutical composition comprises sodium isobutyrate and sodium butyrate. In some embodiments, a pharmaceutical composition comprises sodium butyrate and sodium valerate. In some embodiments, a pharmaceutical composition comprises sodium butyrate and sodium isovalerate.

In some embodiments, a pharmaceutical composition comprises butyric acid and one additional SCFA. In some embodiments, a pharmaceutical composition comprises propionic acid and butyric acid. In some embodiments, a pharmaceutical composition comprises isobutyric acid and butyric acid. In some embodiments, a pharmaceutical composition comprises butyric acid and valeric acid. In some embodiments, a pharmaceutical composition comprises butyric acid and isovaleric acid.

In some embodiments, a pharmaceutical composition comprises isobutyric acid or a pharmaceutically-acceptable salt thereof and one additional SCFA. In some embodiments, a pharmaceutical composition comprises isobutyric acid or a pharmaceutically-acceptable salt thereof and propionic acid or a pharmaceutically-acceptable salt thereof. In some embodiments, a pharmaceutical composition comprises isobutyric acid or a pharmaceutically-acceptable salt thereof and valerate or a pharmaceutically-acceptable salt thereof. In some embodiments, a pharmaceutical composition comprises isobutyric acid or a pharmaceutically-acceptable salt thereof and isovalerate or a pharmaceutically-acceptable salt thereof.

In some embodiments, a pharmaceutical composition comprises sodium isobutyrate and one additional SCFA. In some embodiments, a pharmaceutical composition comprises sodium isobutyrate and sodium propionate. In some embodiments, a pharmaceutical composition comprises sodium isobutyrate and sodium valerate. In some embodiments, a pharmaceutical composition comprises sodium isobutyrate and sodium isovalerate.

In some embodiments, a pharmaceutical composition comprises isobutyric acid and one additional SCFA. In some embodiments, a pharmaceutical composition comprises isobutyric acid and propionic acid. In some embodiments, a pharmaceutical composition comprises isobutyric acid and valeric acid. In some embodiments, a pharmaceutical composition comprises isobutyric acid and isovaleric acid.

In some embodiments, a pharmaceutical composition comprises from about 1.5 g to about 2 g of butyric acid or a pharmaceutically-acceptable salt thereof and from about 150 mg to about 200 mg of propionic acid or a pharmaceutically-acceptable salt thereof. In some embodiments, a pharmaceutical composition comprises about 4 g of butyric acid or a pharmaceutically-acceptable salt thereof and from about 250 mg of propionic acid or a pharmaceutically-acceptable salt thereof. In some embodiments, a pharmaceutical composition comprises from about 3.5 g to about 4 g of butyric acid or a pharmaceutically-acceptable salt thereof and from about 150 mg to about 300 mg of propionic acid or a pharmaceutically-acceptable salt thereof. In some embodiments, a pharmaceutical composition comprises from about 2.5 g to about 3 g of butyric acid or a pharmaceutically-acceptable salt thereof and from about 300 mg to about 400 mg of propionic acid or a pharmaceutically-acceptable salt thereof.

In some embodiments, a pharmaceutical composition comprises 250 mg sodium propionate and 4,000 mg sodium isobutyrate. In some embodiments, a pharmaceutical composition comprises 250 mg sodium valerate and 4,000 mg sodium isobutyrate. In some embodiments, a pharmaceutical composition comprises 250 mg sodium isovalerate and 4,000 mg sodium isobutyrate.

In some embodiments, a pharmaceutical composition comprises 250 mg propionic acid and 4,000 mg isobutyric acid. In some embodiments, a pharmaceutical composition comprises 250 mg valeric acid and 4,000 mg isobutyric acid. In some embodiments, a pharmaceutical composition comprises 250 mg isovaleric acid and 4,000 mg isobutyric acid.

### Methods of Administration

Pharmaceutical compositions or therapeutic agents described herein can be administered before, during, or after the occurrence of a disease or condition, and the timing of administering the composition containing a therapeutic agent can vary. For example, the pharmaceutical compositions or therapeutic agents can be used as a prophylactic and can be administered continuously to subjects with a propensity to conditions or diseases to lessen a likelihood of the occurrence of the disease or condition. The pharmaceutical compositions or therapeutic agents can be administered to a subject during or as soon as possible after the onset of the symptoms. The administration of the pharmaceutical compositions or therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

In some embodiments, pharmaceutical compositions or therapeutic agents can be administered to a patient exhibiting early symptoms of an illness. In some embodiments, the symptom is coughing. In some embodiments, the symptom is fever. Pharmaceutical compositions or therapeutic agents can be administered as soon as is practical after the onset of a disease or condition is detected or suspected, and for a length of time necessary for the treatment of the disease, such as, for example, from about 1 month to about 3 months. In some embodiments, the length of time pharmaceutical compositions or therapeutic agents can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Multiple pharmaceutical compositions or therapeutic agents can be administered in any order or simultaneously. In some embodiments, a pharmaceutical composition of the disclosure is administered in combination with, before, or after treatment with another therapeutic agent. If simultaneously, the pharmaceutical compositions or therapeutic agents can be provided in a single, unified form, or in multiple forms, for example, as multiple separate pills. The pharmaceutical compositions or therapeutic agents can be packed together or separately, in a single package or in a plurality of packages. One or all of the pharmaceutical composition or therapeutic agents can be given in multiple doses. If not simultaneous, the timing between the multiple doses can vary to as much as about a month.

In some embodiments, a pharmaceutical composition is administered in a daily oral dose. In some embodiments, a pharmaceutical composition is administered in a daily oral dose 3 times a day. In some embodiments, a formulation is administered in a daily oral dose 3 times a day for one week. In some embodiments, a formulation is administered in a daily oral dose 3 times a day for two weeks. In some embodiments, a formulation is administered in a daily oral dose 3 times a day for three weeks.

In some embodiments, a pharmaceutical composition is administered in a daily oral dose with food. In some embodiments, a pharmaceutical composition is administered in a daily oral dose 3 times a day, each time with food. In some embodiments, a formulation is administered in a daily oral dose 3 times a day for one week, each time with food. In some embodiments, a formulation is administered in a daily oral dose 3 times a day for two weeks, each time with food. In some embodiments, a formulation is administered in a daily oral dose 3 times a day for three weeks, each time with food.

In some embodiments, a pharmaceutical composition is administered in a daily oral dose after food. In some embodiments, a pharmaceutical composition is administered in a daily oral dose 3 times a day, each time after food. In some embodiments, a formulation is administered in a daily oral dose 3 times a day for one week, each time after food. In some embodiments, a formulation is administered in a daily oral dose 3 times a day for two weeks, each time after food. In some embodiments, a formulation is administered in a daily oral dose 3 times a day for three weeks, each time after food. In some embodiments, a formulation is administered about 5 minutes, about 15 minutes, about 30 minutes, or about an hour after food.

In some embodiments, a pharmaceutical composition is administered in a daily oral dose. In some embodiments, a pharmaceutical composition is administered in a daily oral dose 3 times a day. In some embodiments, a formulation is administered in a daily oral dose 3 times a day for one month. In some embodiments, a formulation is administered in a daily oral dose 3 times a day for two months. In some embodiments, a formulation is administered in a daily oral dose 3 times a day for three months. In some embodiments, a formulation is administered in a daily oral dose 3 times a day for one year. In some embodiments, a formulation is administered in a daily oral dose 3 times a day for two years. In some embodiments, a formulation is administered in a daily oral dose 3 times a day for three years.

In some embodiments, a pharmaceutical composition is administered in a daily oral dose with food. In some embodiments, a pharmaceutical composition is administered in a daily oral dose 3 times a day, each time with food. In some embodiments, a formulation is administered in a daily oral dose 3 times a day for one month, each time with food. In some embodiments, a formulation is administered in a daily oral dose 3 times a day for two months, each time with food. In some embodiments, a formulation is administered in a daily oral dose 3 times a day for three months, each time with food. In some embodiments, a formulation is administered in a daily oral dose 3 times a day for one year, each time with food. In some embodiments, a formulation is administered in a daily oral dose 3 times a day for two years, each time with food. In some embodiments, a formulation is administered in a daily oral dose 3 times a day for three years, each time with food.

In some embodiments, a pharmaceutical composition is administered in a daily oral dose after food. In some embodiments, a pharmaceutical composition is administered in a daily oral dose 3 times a day, each time after food. In some embodiments, a formulation is administered in a daily oral dose 3 times a day for one month, each time after food. In some embodiments, a formulation is administered in a daily oral dose 3 times a day for two months, each time after food. In some embodiments, a formulation is administered in a daily oral dose 3 times a day for three months, each time after food. In some embodiments, a formulation is administered in a daily oral dose 3 times a day for one year, each time after food. In some embodiments, a formulation is administered in a daily oral dose 3 times a day for two years, each time after food. In some embodiments, a formulation is administered in a daily oral dose 3 times a day for three years, each time after food. In some embodiments, a formulation is administered about 5 minutes, about 15 minutes, about 30 minutes, or about an hour after food.

In some embodiments, a pharmaceutical composition comprising butyric acid or a pharmaceutically-acceptable salt thereof and propionic acid or a pharmaceutically-acceptable salt thereof is administered in a daily oral dose. In some embodiments, a pharmaceutical composition comprising butyric acid or a pharmaceutically-acceptable salt thereof and propionic acid or a pharmaceutically-acceptable salt thereof is administered in a daily oral dose 3 times a day. In some embodiments, a pharmaceutical composition comprising butyric acid or a pharmaceutically-acceptable salt thereof and propionic acid or a pharmaceutically-acceptable salt thereof is administered in a daily oral dose 3 times a day. In some embodiments, a pharmaceutical composition comprising butyric acid or a pharmaceutically-acceptable salt thereof and propionic acid or a pharmaceutically-acceptable salt thereof is administered in a daily oral dose 3 times a day for one week. In some embodiments, a pharmaceutical composition comprising butyric acid or a pharmaceutically-acceptable salt thereof and propionic acid or a pharmaceutically-acceptable salt thereof is administered in a daily oral dose 3 times a day for two weeks. In some embodiments, a pharmaceutical composition comprising butyric acid or a pharmaceutically-acceptable salt thereof and propionic acid or a pharmaceutically-acceptable salt thereof is administered in a daily oral dose 3 times a day for three weeks.

In some embodiments, a pharmaceutical composition comprising butyric acid or a pharmaceutically-acceptable salt thereof and propionic acid or a pharmaceutically-acceptable salt thereof is administered in a daily oral dose with food. In some embodiments, a pharmaceutical composition comprising butyric acid or a pharmaceutically-acceptable salt thereof and propionic acid or a pharmaceutically-acceptable salt thereof is administered in a daily oral dose 3 times a day, each time with food. In some embodiments, a pharmaceutical composition comprising butyric acid or a pharmaceutically-acceptable salt thereof and propionic acid or a pharmaceutically-acceptable salt thereof is administered in a daily oral dose 3 times a day, each time with food. In some embodiments, a pharmaceutical composition comprising butyric acid or a pharmaceutically-acceptable salt thereof and propionic acid or a pharmaceutically-acceptable salt thereof is administered in a daily oral dose 3 times a day for one week, each time with food. In some embodiments, a pharmaceutical composition comprising butyric acid or a pharmaceutically-acceptable salt thereof and propionic acid or a pharmaceutically-acceptable salt thereof is administered in a daily oral dose 3 times a day for two weeks, each time with food. In some embodiments, a pharmaceutical composition comprising butyric acid or a pharmaceutically-acceptable salt thereof and propionic acid or a pharmaceutically-acceptable salt thereof is administered in a daily oral dose 3 times a day for three weeks, each time with food.

In some embodiments, a pharmaceutical composition comprising butyric acid or a pharmaceutically-acceptable salt thereof and propionic acid or a pharmaceutically-acceptable salt thereof is administered in a daily oral dose after food. In some embodiments, a pharmaceutical composition comprising butyric acid or a pharmaceutically-acceptable salt thereof and propionic acid or a pharmaceutically-acceptable salt thereof is administered in a daily oral dose 3 times a day, each time after food. In some embodiments, a pharmaceutical composition comprising butyric acid or a pharmaceutically-acceptable salt thereof and propionic acid or a pharmaceutically-acceptable salt thereof is administered in a daily oral dose 3 times a day, each time after food. In some embodiments, a pharmaceutical composition comprising butyric acid or a pharmaceutically-acceptable salt thereof and propionic acid or a pharmaceutically-acceptable salt thereof is administered in a daily oral dose 3 times a day for one week, each time after food. In some embodiments, a pharmaceutical composition comprising butyric acid or a pharmaceutically-acceptable salt thereof and propionic acid or a pharmaceutically-acceptable salt thereof is administered in a daily oral dose 3 times a day for two weeks, each time after food. In some embodiments, a pharmaceutical composition comprising butyric acid or a pharmaceutically-acceptable salt thereof and propionic acid or a pharmaceutically-acceptable salt thereof is administered in a daily oral dose 3 times a day for three weeks, each time after food. In some embodiments, a formulation is administered about 5 minutes, about 15 minutes, about 30 minutes, or about an hour after food.

In some embodiments, a pharmaceutical composition comprising sodium butyrate and sodium propionate is administered in a daily oral dose. In some embodiments, a pharmaceutical composition comprising sodium butyrate and sodium propionate is administered in a daily oral dose 3 times a day. In some embodiments, a pharmaceutical composition comprising sodium butyrate and sodium propionate is administered in a daily oral dose 3 times a day for one week. In some embodiments, a pharmaceutical composition comprising sodium butyrate and sodium propionate is administered in a daily oral dose 3 times a day for two weeks. In some embodiments, a pharmaceutical composition comprising sodium butyrate and sodium propionate is administered in a daily oral dose 3 times a day for three weeks.

In some embodiments, a pharmaceutical composition comprising sodium butyrate and sodium propionate is administered in a daily oral dose with food. In some embodiments, a pharmaceutical composition comprising sodium butyrate and sodium propionate is administered in a daily oral dose 3 times a day, each time with food. In some embodiments, a pharmaceutical composition comprising sodium butyrate and sodium propionate is administered in a daily oral dose 3 times a day for one week, each time with food. In some embodiments, a pharmaceutical composition comprising sodium butyrate and sodium propionate is administered in a daily oral dose 3 times a day for two weeks, each time with food. In some embodiments, a pharmaceutical composition comprising sodium butyrate and sodium propionate is administered in a daily oral dose 3 times a day for three weeks, each time with food.

In some embodiments, a pharmaceutical composition comprising sodium butyrate and sodium propionate is administered in a daily oral dose after food. In some embodiments, a pharmaceutical composition comprising sodium butyrate and sodium propionate is administered in a daily oral dose 3 times a day, each time after food. In some embodiments, a pharmaceutical composition comprising sodium butyrate and sodium propionate is administered in a daily oral dose 3 times a day for one week, each time after food. In some embodiments, a pharmaceutical composition comprising sodium butyrate and sodium propionate is administered in a daily oral dose 3 times a day for two weeks, each time after food. In some embodiments, a pharmaceutical composition comprising sodium butyrate and sodium propionate is administered in a daily oral dose 3 times a day for three weeks, each time after food. In some embodiments, a formulation is administered about 5 minutes, about 15 minutes, about 30 minutes, or about an hour after food.

In some embodiments, a pharmaceutical composition comprising butyric acid and propionic acid, or a pharmaceutically-acceptable salt of each, is administered in a daily oral dose. In some embodiments, a pharmaceutical composition comprising butyric acid and propionic acid, or a pharmaceutically-acceptable salt of each, is administered in a daily oral dose 3 times a day. In some embodiments, a pharmaceutical composition comprising butyric acid and propionic acid, or a pharmaceutically-acceptable salt of each, is administered in a daily oral dose 3 times a day. In some embodiments, a pharmaceutical composition comprising butyric acid and propionic acid, or a pharmaceutically-acceptable salt of each, is administered in a daily oral dose 3 times a day for one week. In some embodiments, a pharmaceutical composition comprising butyric acid and propionic acid, or a pharmaceutically-acceptable salt of each, is administered in a daily oral dose 3 times a day for two weeks. In some embodiments, a pharmaceutical composition comprising butyric acid and propionic acid, or a pharmaceutically-acceptable salt of each, is administered in a daily oral dose 3 times a day for three weeks.

In some embodiments, a pharmaceutical composition comprising butyric acid and propionic acid, or a pharmaceutically-acceptable salt of each, is administered in a daily oral dose with food. In some embodiments, a pharmaceutical composition comprising butyric acid and propionic acid, or a pharmaceutically-acceptable salt of each, is administered in a daily oral dose 3 times a day, each time with food. In some embodiments, a pharmaceutical composition comprising butyric acid and propionic acid, or a pharmaceutically-acceptable salt of each, is administered in a daily oral dose 3 times a day, each time with food. In some embodiments, a pharmaceutical composition comprising butyric acid and propionic acid, or a pharmaceutically-acceptable salt of each, is administered in a daily oral dose 3 times a day for one week, each time with food. In some embodiments, a pharmaceutical composition comprising butyric acid and propionic acid, or a pharmaceutically-acceptable salt of each, is administered in a daily oral dose 3 times a day for two weeks, each time with food. In some embodiments, a pharmaceutical composition comprising butyric acid and propionic acid, or a pharmaceutically-acceptable salt of each, is administered in a daily oral dose 3 times a day for three weeks, each time with food.

In some embodiments, a pharmaceutical composition comprising butyric acid and propionic acid, or a pharmaceutically-acceptable salt of each, is administered in a daily oral dose after food. In some embodiments, a pharmaceutical composition comprising butyric acid and propionic acid, or a pharmaceutically-acceptable salt of each, is administered in a daily oral dose 3 times a day, each time after food. In some embodiments, a pharmaceutical composition comprising butyric acid and propionic acid, or a pharmaceutically-acceptable salt of each, is administered in a daily oral dose 3 times a day, each time after food. In some embodiments, a pharmaceutical composition comprising butyric acid and propionic acid, or a pharmaceutically-acceptable salt of each, is administered in a daily oral dose 3 times a day for one week, each time after food. In some embodiments, a pharmaceutical composition comprising butyric acid and propionic acid, or a pharmaceutically-acceptable salt of each, is administered in a daily oral dose 3 times a day for two weeks, each time after food. In some embodiments, a pharmaceutical composition comprising butyric acid and propionic acid, or a pharmaceutically-acceptable salt of each, is administered in a daily oral dose 3 times a day for three weeks, each time after food. In some embodiments, a formulation is administered about 5 minutes, about 15 minutes, about 30 minutes, or about an hour after food.

In some embodiments, a pharmaceutical composition is administered in a daily parenteral dose. In some embodiments, a pharmaceutical composition is administered in a daily parenteral dose 3 times a day. In some embodiments, a formulation is administered in a daily parenteral dose 3 times a day for one week. In some embodiments, a formulation is administered in a daily parenteral dose 3 times a day for two weeks. In some embodiments, a formulation is administered in a daily parenteral dose 3 times a day for three weeks.

In some embodiments, a pharmaceutical composition is administered in a daily parenteral dose. In some embodiments, a pharmaceutical composition is administered in a daily parenteral dose 3 times a day. In some embodiments, a formulation is administered in a daily parenteral dose 3 times a day for one month. In some embodiments, a formulation is administered in a daily parenteral dose 3 times a day for two months. In some embodiments, a formulation is administered in a daily parenteral dose 3 times a day for three months. In some embodiments, a formulation is administered in a daily parenteral dose 3 times a day for one year. In some embodiments, a formulation is administered in a daily parenteral dose 3 times a day for two years. In some embodiments, a formulation is administered in a daily parenteral dose 3 times a day for three years.

In some embodiments, a pharmaceutical composition comprising butyric acid or a pharmaceutically-acceptable salt thereof and propionic acid or a pharmaceutically-acceptable salt thereof is administered in a daily parenteral dose. In some embodiments, a pharmaceutical composition comprising butyric acid or a pharmaceutically-acceptable salt thereof and propionic acid or a pharmaceutically-acceptable salt thereof is administered in a daily parenteral dose 3 times a day. In some embodiments, a pharmaceutical composition comprising butyric acid or a pharmaceutically-acceptable salt thereof and propionic acid or a pharmaceutically-acceptable salt thereof is administered in a daily parenteral dose 3 times a day for one week. In some embodiments, a pharmaceutical composition comprising butyric acid or a pharmaceutically-acceptable salt thereof and propionic acid or a pharmaceutically-acceptable salt thereof is administered in a daily parenteral dose 3 times a day for two weeks. In some embodiments, a pharmaceutical composition comprising butyric acid or a pharmaceutically-acceptable salt thereof and propionic acid or a pharmaceutically-acceptable salt thereof is administered in a daily parenteral dose 3 times a day for three weeks.

In some embodiments, a pharmaceutical composition comprising sodium butyrate and sodium propionate is administered in a daily parenteral dose. In some embodiments, a pharmaceutical composition comprising sodium butyrate and sodium propionate is administered in a daily parenteral dose 3 times a day. In some embodiments, a pharmaceutical composition comprising sodium butyrate and sodium propionate is administered in a daily parenteral dose 3 times a day for one week. In some embodiments, a pharmaceutical composition comprising sodium butyrate and sodium propionate is administered in a daily parenteral dose 3 times a day for two weeks. In some embodiments, a pharmaceutical composition comprising sodium butyrate and sodium propionate is administered in a daily parenteral dose 3 times a day for three weeks.

In some embodiments, a pharmaceutical composition comprising butyric acid and propionic acid, or a pharmaceutically-acceptable salt of each, is administered in a daily parenteral dose. In some embodiments, a pharmaceutical composition comprising butyric acid and propionic acid, or a pharmaceutically-acceptable salt of each, is administered in a daily parenteral dose 3 times a day. In some embodiments, a pharmaceutical composition comprising butyric acid and propionic acid, or a pharmaceutically-acceptable salt of each, is administered in a daily parenteral dose 3 times a day for one week. In some embodiments, a pharmaceutical composition comprising butyric acid and propionic acid, or a pharmaceutically-acceptable salt of each, is administered in a daily parenteral dose 3 times a day for two weeks. In some embodiments, a pharmaceutical composition comprising butyric acid and propionic acid, or a pharmaceutically-acceptable salt of each, is administered in a daily parenteral dose 3 times a day for three weeks.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

A pharmaceutical composition of the disclosure can be used, for example, before, during, or after treatment of a subject with, for example, another pharmaceutical agent. In some embodiments, a pharmaceutical composition of the disclosure is administered with an antiviral agent. In some embodiments, a pharmaceutical composition of the disclosure is administered with an antibiotic agent. Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

A therapeutically-effective amount can vary widely depending on the severity of the disease, the age and relative health of the subject, the potency of the compounds used, and other factors. The compounds can be used singly or in combination with one or more therapeutic agents as components of mixtures. In some embodiments, the compounds can be used in combination with 1, 2, 3, 4, 5, 6, 7, 8, 9, or 10 additional therapeutic agents. In some embodiments, the compounds of the disclosure can be used with 1 additional therapeutic agent. In some embodiments, the compounds of the disclosure can be used with 2 additional therapeutic agents. In some embodiments, the compounds of the disclosure can be used with 3 additional therapeutic agents. In some embodiments, a pharmaceutical composition of the disclosure is administered with an antiviral agent. In some embodiments, a pharmaceutical composition of the disclosure is administered with an antibiotic agent.

### Methods of Treatment

In some embodiments, the compositions of the disclosure can be used to treat a condition. In some embodiments, the condition is colitis-associated colorectal cancer (CRC). In some embodiments, the condition is hepatocellular carcinoma.

### Kits

Compositions of the disclosure can be packaged as a kit. In some embodiments, a kit includes written instructions on the administration or use of the composition. The written material can be, for example, a label. The written material can suggest conditional methods of administration. The instructions provide the subject and the supervising physician with the best guidance for achieving the optimal clinical outcome from the administration of the therapy. The written material can be a label. In some embodiments, the label can be approved by a regulatory agency, for example, the U.S. Food and Drug Administration (FDA), the European Medicines Agency (EMA), or other regulatory agencies.

### EXAMPLES

### Example 1

### SCFA Treatment Reduces the Number of Dysplastic and HCC Nodules

SCFAs were used to treat colitis-associated colorectal cancer, which develops on a background of chronic inflammation, as does HCC from CLD. Thus, experiments were designed to test the hypothesis that SCFAs delays the development of dysplastic nodules and/or HCC in an HBx transgenic (HBxTg) mouse model that closely recapitulates many steps in the pathogenesis of CLD and HCC seen in human HBV carriers. To determine the impact of SCFAs on the development of dysplastic nodules, HBxTg mice were treated with SCFAs or PBS from 6-9 months of age (henceforth referred to as ‘9-month group’). To determine the impact of SCFAs upon HCC development, an additional group of mice was treated with SCFAs or PBS from 9-12 months of age (henceforth referred to as ‘12-month group’).

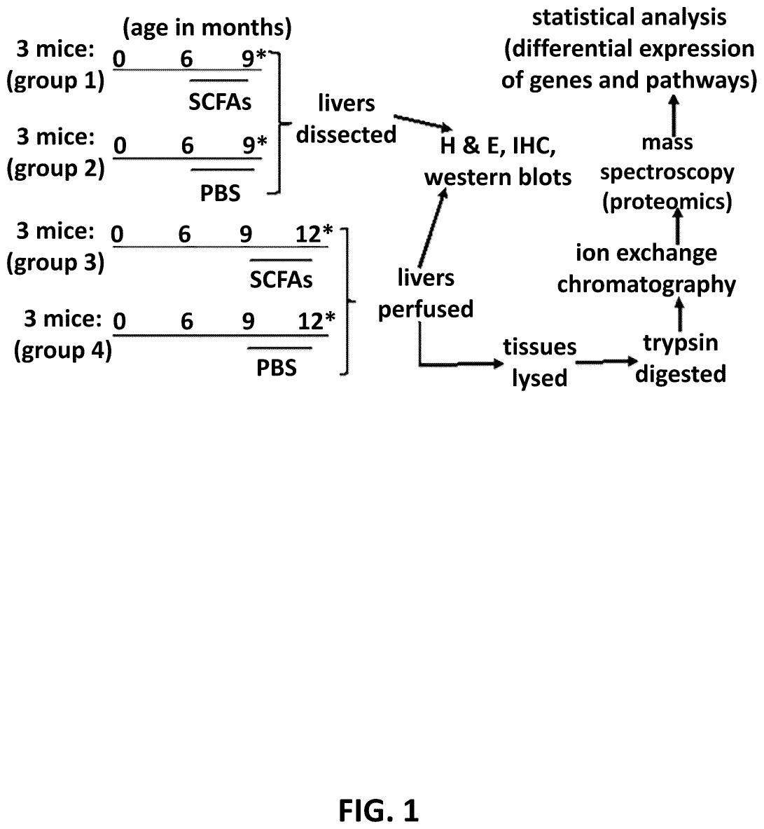

At the end of treatment, livers from 9-month and 12-month groups were evaluated for histopathology and 12-month group livers further analyzed by proteomics (FIG. 1). There was no statistical difference in the number of mice that developed hepatitis or steatosis in SCFA-treated compared to PBS control mice in either age group (TABLES 1 and 2). This result was expected since the mice developed these disease stages before treatment was initiated. In contrast, significantly fewer SCFA-treated mice developed dysplasia in both the 9-month (P<0.02) and 12-month groups (P<0.05).

TABLE 1 shows liver histopathology in 9-month old mice after three months of treatment. TABLE 2 shows liver histopathology in 12-month old mice after three months of treatment. Livers were assessed for hepatitis, steatosis, dysplasia, and HCC in 5 different sections from different parts of each lobe. At this age, HCC nodules were not visible. Plus sign (+) indicates the presence of disease stage and the minus sign (−) indicates absence. The number of lesions were counted and listed in parentheses.

TABLE 1

| | No. of | | | | HCC (total |
|---|---|---|---|---|---|
| Group | mice | hepatitis | steatosis | dysplasia | no. of tumors) |
| PBS | 2 | + | + | + | + (1) |
| (control) | 1 | + | − | + | + (2) |
| | 2 | + | − | + | − |
| | 9 | − | − | + | − |
| | 1 | + | + | − | − |
| | 1 | + | + | + | − |
| | 6 | − | + | + | − |
| | 2 | − | − | − | − |
| SCFAs | 2 | + | + | + | + (2) |
| | 6 | + | + | + | − |
| | 1 | + | + | − | − |
| | 2 | − | + | + | − |
| | 1 | − | + | − | − |
| | 6 | − | − | − | − |

TABLE 2

| | No. of | | | | HCC/(total |
|---|---|---|---|---|---|
| Group | mice | Hepatitis | steatosis | dysplasia | no. of tumors) |
| PBS | 9 | + | + | + | + (34) |
| (control) | 5 | − | + | + | + (12) |
| | 2 | + | − | + | + (2) |
| | 1 | − | − | + | + (6) |
| | 1 | + | + | + | − |
| | 3 | − | + | + | − |
| | 2 | + | − | + | − |
| | 4 | − | − | + | − |
| | 1 | + | − | − | − |
| | 1 | − | − | − | − |
| SCFAs | 4 | + | + | + | + (8) |
| | 1 | − | + | + | + (4) |
| | 3 | − | + | + | − |
| | 1 | + | − | − | − |
| | 4 | + | + | + | − |
| | 3 | − | − | + | − |
| | 2 | + | − | + | − |
| | 6 | − | − | − | − |

Further, SCFAs reduced the number of mice that developed HCC compared to control mice in the 12-month group (TABLE 3, P<0.001). TABLE 3 shows tumors in SCFA-treated and control mice at 9-months and 12-months of age.

TABLE 3

| | | | No. and % of | | | |
|---|---|---|---|---|---|---|
| | No. | | mice with | | No. and % of mice | |
| Age | of | | dysplastic | | with visible HCC | |
| (mo.) | mice | Treatment | nodules | | nodules | |
| 9 | 24 | PBS | 21 | (87.5%)* | 3 | (12.5%) |
| | 18 | SCFAs | 10 | (55%) | 2 | (11%) |
| 12 | 29 | PBS | 27 | (93%)*** | 17 | (46%)** |
| | 24 | SCFAs | 17 | (71%) | 2 | (8%) |

*P < 0.02;
**P < 0.001;
***P < 0.05.
PBS, phosphate buffered saline; SCFAs, short chain fatty acids.

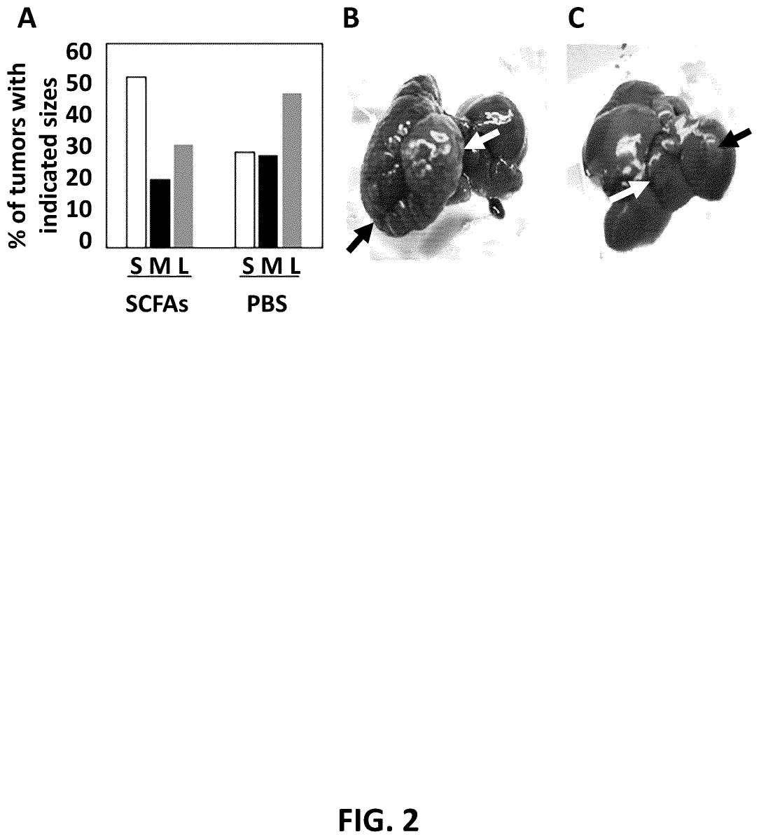

Among those mice that developed tumors, SCFA-treated mice had predominantly small sized tumors compared to predominately large tumors that developed in PBS treated mice (FIG. 2, P<0.001). FIG. 2, Panel A shows the percentage of tumor nodules from the group of 12-month old mice treated with SCFAs or with PBS relative to tumor size. S: small tumors (<0.5 cm), M: medium size tumors (0.5-1 cm), L: large tumors (>1 cm). Panel B shows an example of a large tumor from a PBS-treated mouse. Panel C shows an example of two small tumors from a SCFA-treated mouse. Arrows in (B) and (C) denote the position of the tumor nodules. PBS, phosphate buffered saline; SCFAs, short chain fatty acids.

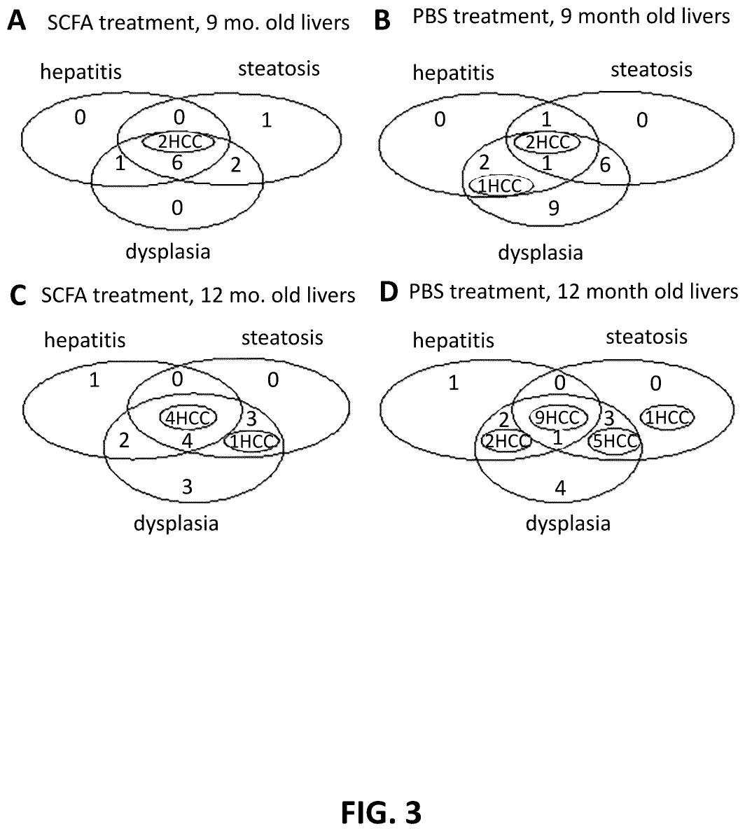

When liver sections from 9-month and 12-month old mice were examined for histopathology by light microscopy, HCC was only present in the livers of mice that also had other lesions characteristic of CLD (FIG. 3, TABLES 1 AND 2), as in human carriers with HCC. Thus, although HCC develops on a background of CLD in both treated and control mice, SCFAs delay the pathogenesis of HBV-associated HCC. FIG. 3 shows the number of mice with the indicated pathology in livers evaluated at 9 months (Panels A and B) and 12 months (Panels C and D). Comparisons are made at each age group between SCFA treated (Panels A and C) and PBS (Panels B and D) treated mice. HCC, hepatocellular carcinoma; PBS, phosphate buffered saline; SCFAs, short chain fatty acids.

### Example 2

### SCFAs Specifically Reduce Cancer Cell Viability

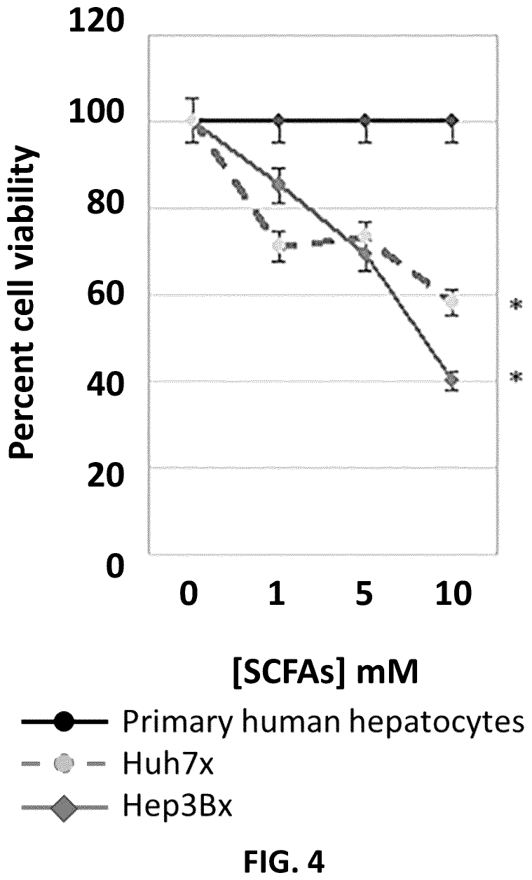

Since SCFAs were able to delay tumor development in the HBxTg mice, additional experiments were designed to evaluate whether SCFAs had an impact on human HCC cell viability. When previously described Hep3Bx and Huh7x human hepatoma cell lines constitutively expressing HBx were treated with SCFAs, cell viability decreased in a dose-dependent manner over a 24-hour period. In contrast, the viability of primary human hepatocytes was unaffected by SCFA treatment at the same doses and time-period (P<0.01; FIG. 4). These observations were consistent with the in vivo experiments, thereby demonstrating one way that SCFA treatment partially inhibited tumor growth.

FIG. 4 shows the effect of SCFAs on primary human hepatocytes and two HBx expressing human HCC cell lines. Primary human hepatocytes and the HCC cell lines transfected with HBx (Hep3Bx and Huh7x) were treated with increasing concentrations of SCFAs and assessed for cell viability using MTS assay. Primary human hepatocytes (●), Huh7x (- [symbol] -), and Hep3Bx ( [symbol] ). All measurements were performed in triplicate. Results are expressed as the percent viability of SCFA-treated compared to PBS-treated cells. * indicates p<0.01. SCFAs, short chain fatty acids.

### Example 3

### Differential Expression of Proteins by Proteomics

Mass spectrometry-based proteomics was performed on SCFA-treated and control 12-month old HBxTg mouse livers to determine the effect of SCFAs on protein expression in biological processes and signaling pathways at the age when tumors appear. Accordingly, three biological replicates were included in each group analyzed. Tissue from different lobes of the liver were taken from each sample. Although these may have included microscopic tumors, the vast majority of the cells in these samples were non-tumor. Among the more than 3,000 proteins identified, 222 proteins were differentially expressed in the 12-month group. Differentially expressed proteins include those detected in SCFA compared to PBS-treated groups at levels significantly different from one another (P<0.05), as well as proteins detected in the majority or all samples in one group and not detected in any samples of the comparison group. The mass spectrometer used in this study, Q exactive, is capable of detecting proteins present in as little as 1 ng of sample, making it a very sensitive mass spectrometer with a low limit of detection.

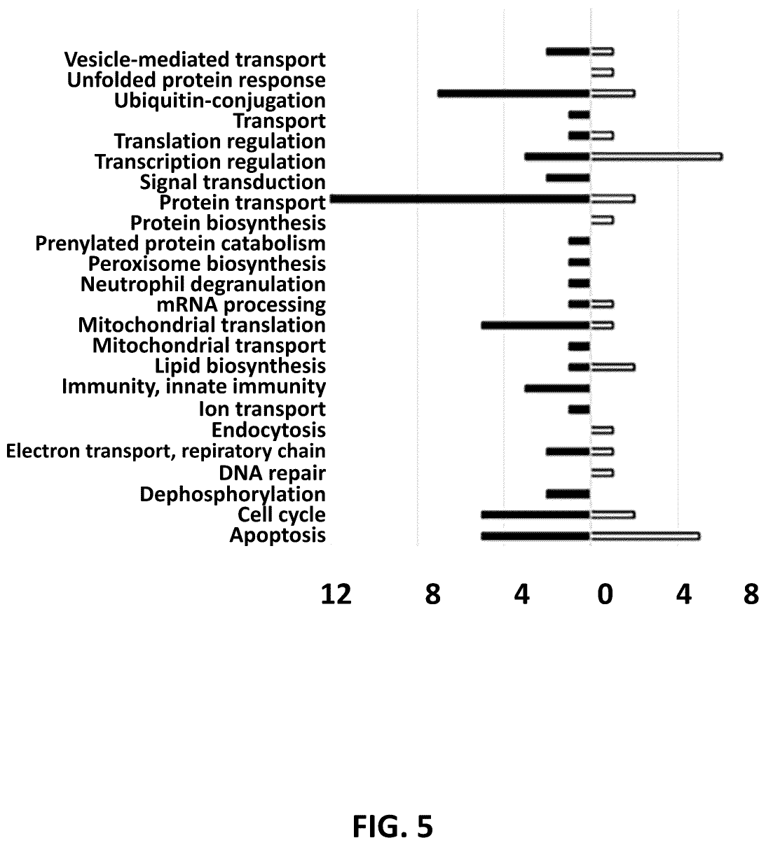

Differentially expressed proteins in livers of SCFA-treated compared to PBS-treated mice were arranged by their GO biological processes (FIG. 5). FIG. 5 shows the number of proteins with decreased (black bars) or increased (gray bars) expression in 12-month old livers from HBxTg mice after SCFA treatment compared to PBS controls arranged by GO biological processes. HBxTg, hepatitis Bx transgenic; PBS, phosphate buffered saline; SCFAs, short chain fatty acids.

Among the 14 differentially expressed genes that mediate protein transport, 13 of them were detectable in the control livers, but were below the limit of detection in the SCFA-treated samples. This observation suggests that SCFAs down-regulate protein transport (TABLE 4), including down-regulated expression of proteins involved in trafficking between the Golgi and endosome (MON), a protein regulating exocytosis (exocyst complex components 2 and 5), nuclear import and export (importin-5, exportin-7, importin subunit α-1, nuclear pore complex protein Nup98), and NF-KB signaling (ELKS/Rab6 interacting CAST) family member.

TABLE 4 shows differentially expressed proteins associated with biological processes that are altered by SCFAs compared to PBS in the 12-month old liver

TABLE 4

| | | Expression in |
|---|---|---|
| | | treated relative to |
| Biological process | Gene name | control |
| Apoptosis | STE20-like serine/threonine-protein kinase | + |
| | Probable ATP-dependent RNA helicase DDX47 | + |
| | Disabled homolog 2 (DAB2) | + |
| | Cyclin-dependent-like kinase 5 | − |
| | Programmed cell death protein 10 | − |
| | Dual serine/threonine and tyrosine protein kinase | + |
| | Baculoviral IAP repeat-containing protein 6 | + |
| | Nck-associated protein 1 | − |
| | Beta-catenin-like protein 1 | − |
| | Protein prune homolog | − |
| Cell cycle | Centromere/kinetochore protein zw10 homolog | − |
| | Cyclin-dependent-like kinase 5 | − |
| | DCC-interacting protein 13-alpha | − |
| | Baculoviral IAP repeat-containing protein 6 | + |
| | BRISC complex subunit Abro1 | + |
| | Growth arrest and DNA damage-(GADD) | − |
| | inducible proteins-interacting protein 1 | |
| | Charged multivesicular body protein 1b-1 | − |
| Dephosphorylation | Haloacid dehalogenase-like hydrolase domain- | −3 |
| | containing protein 2 | |
| | Dual specificity protein phosphatase 23 | − |
| DNA repair | Actin-like protein 6A | + |
| Electron transport, | Cytochrome c oxidase subunit 1 | + |
| respiratory chain | Cytochrome b-c1 complex subunit 8 | − |
| | Protein SCO2 homolog, mitochondrial | − |
| Endocytosis | Disabled homolog 2 | + |
| Ion transport | V-type proton ATPase catalytic subunit A | −2.3 |
| Immunity, Innate | Chitinase domain-containing protein 1 | − |
| immunity | Complement C5 | − |
| | Protein jagunal homolog 1 | − |
| Lipid biosynthesis | Choline/ethanolamine phosphotransferase 1 | + |
| | CDP-diacylglycerol-3-phosphatidyltransferase | + |
| | Phosphatidate cytidylyltransferase, mitochondrial | − |
| Mitochondrial | ATP-binding cassette sub-family B member 10, | − |
| transport | mitochondrial | |
| Mitochondrial | 28S ribosomal protein S18b, mitochondrial | + |
| translation | 28S ribosomal protein S7, mitochondrial | − |
| | 39S ribosomal protein L38, mitochondrial | − |
| | 39S ribosomal protein L3, mitochondrial | − |
| | 39S ribosomal protein L53, mitochondrial | − |
| | 28S ribosomal protein, mitochondrial | − |
| mRNA processing | Crooked neck-like protein 1 | + |
| | RNA-binding protein with serine-rich domain 1 | − |
| Neutrophil | N-acetylgalactosamine-6-sulfatase | − |
| degranulation | | |
| Peroxisome | Peroxisomal membrane protein PEX16 | − |
| biosynthesis | | |
| Prenylated protein | Prenylcysteine oxidase | −3.7 |
| catabolism | | |
| Protein | ATP-dependent RNA helicase Dhx29 | + |
| biosynthesis | | |
| Protein transport | Mitochondrial import receptor subunit TOM20 | + |
| | homolog | |
| | ELKS/Rab6-interacting/CAST family member 1 | − |
| | Transcription and mRNA export factor ENY2 | + |
| | Nuclear pore complex protein Nup98 | − |
| | Importin subunit alpha-1 | − |
| | Importin-5 | − |
| | Exportin-7 | − |
| | Golgi SNAP receptor complex member 2 | − |
| | Exocyst complex component 5 | − |
| | Exocyst complex component 2 | − |
| | Protein jagunal homolog 1 | − |
| | Protein MON2 homolog | − |
| | WASH complex subunit strumpellin | − |
| | Charged multivesicular body protein 1b-1 | − |
| Signal | Ribosomal protein S6 kinase beta-2 | − |
| transduction | Protein S100-A11 | − |
| Transcription | Transcription elongation factor A protein 3 | + |
| regulation | Beta-arrestin-1 | − |
| | Transcription and mRNA export factor ENY2 | + |
| | SWI/SNF complex subunit SMARCC2 | + |
| | Actin-like protein 6A | + |
| | Transcription initiation factor TFIID subunit 5 | − |
| | Cryptochrome-1 | − |
| | Leucine-rich repeat flightless-interacting protein 1 | + |
| | Nucleoplasmin-3 | + |
| Translation | Protein quaking | − |
| regulation | Heterogeneous nuclear ribonucleoprotein L-like | + |
| Transport | Aquaporin-9 | − |
| Ubiquitin- | Microtubule-associated proteins 1A/1B light chain | − |
| conjugation | 3B | |
| | Ubiquitin carboxyl-terminal hydrolase 19 | − |
| | Ubiquitin-conjugating enzyme E2 J1 | − |
| | E3 ubiquitin-protein ligase ZNRF2 | − |
| | Ubiquitin fusion degradation protein 1 homolog | −2 |
| | Baculoviral IAP repeat-containing protein 6 | + |
| | BRISC complex subunit Abrol | + |
| | Proteasome inhibitor PI31 subunit | − |
| | NHL repeat-containing protein 3 | − |
| Unfolded protein | Derlin-2 | + |
| response | | |
| Vesicle-mediated | Vacuolar fusion protein CCZ1 homolog | + |
| transport | Dynactin subunit 4 | − |
| | Centromere/kinetochore protein zw10 homolog | − |

*according to uniport GO biological processes.
¹+, proteins that are strongly up-regulated by SCFA treatment and undetectable in PBS controls; −, proteins that are strongly up-regulated in PBS controls and undetectable in SCFA treated livers. Proteins with numerical values represent the fold change in SCFA treated compared to PBS treated mice.
PBS, phosphate buffered saline; SCFAs, short chain fatty acids.

Further, 10 proteins involved in apoptotic pathways were differentially expressed after SCFA treatment. This observation suggests that SCFAs may promote apoptosis (via increased expression of STE20-like ser/thr kinase, ATP-dependent RNA helicase DDX47, and the tumor suppressor DAB2), or protect from apoptosis (via up-regulation of the baculoviral IAP repeat-containing protein 6, down regulation of β-catenin-like protein 1, and the tumor suppressor protein prune homolog). In transcriptional regulation, SCFAs are known transcriptional regulators via histone deacetylase inhibition, and treatment results in the up-regulated expression of transcription elongation factor A protein 3, ENY2, actin-like protein 6A, and leucine-rich repeat flightless-interacting protein 1 (a transcriptional repressor), as well as down-regulated expression of β-arrestin-1, TFIID subunit 5, and cryptochrome-1.

Transcription may also be altered by differential expression of the chromatin remodeling proteins SWI/SNF complex subunit of SMARCC2 and nucleoplasmin-3. The expression of 6 mitochondrial proteins are also altered, being detectable in PBS control livers but undetectable in the livers of SCFA-treated mice. The expression of smaller numbers of proteins are also altered in a variety of other pathways (FIG. 5, TABLE 4) by SCFAs.

Pathway analysis of 12-month old livers showed that SCFA treatment was associated with the downregulation of pathways known to be activated by HBx in hepatocarcinogenesis. These pathways include inflammation, PI3K, PDGF, FGF, IGF, EGF, Wnt, VEGF, and Ras (FIG. 5). Proteins associated with these pathways were up-regulated in PBS fed livers but undetectable in SCFA livers. Aberrant activation of these signaling pathways have all been implicated in HCC initiation and progression. These pathways drive cell proliferation and growth, block apoptosis, and promote angiogenesis, all of which affect the pathogenesis of HCC.

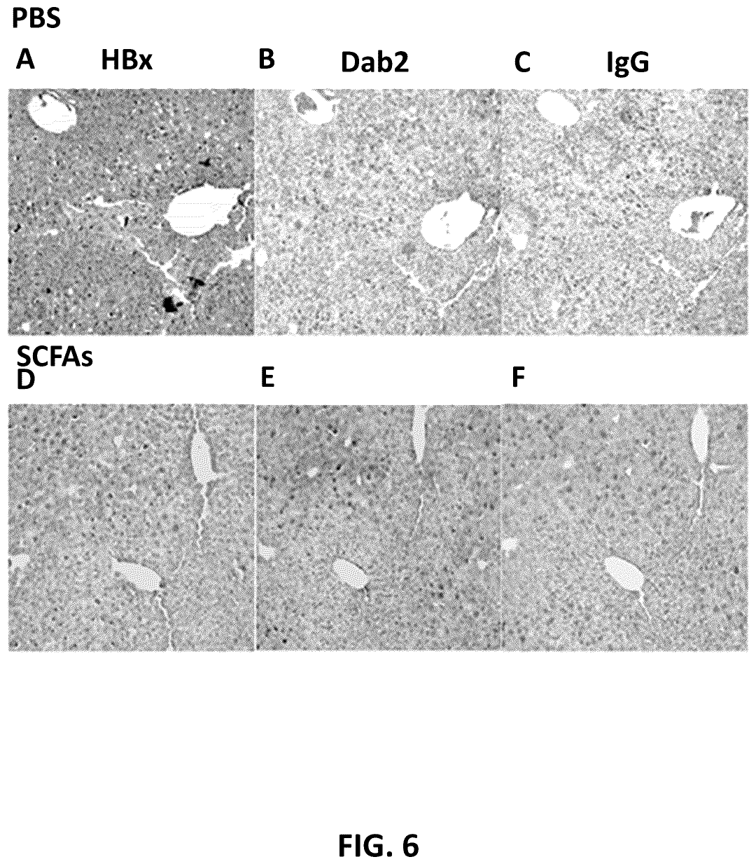

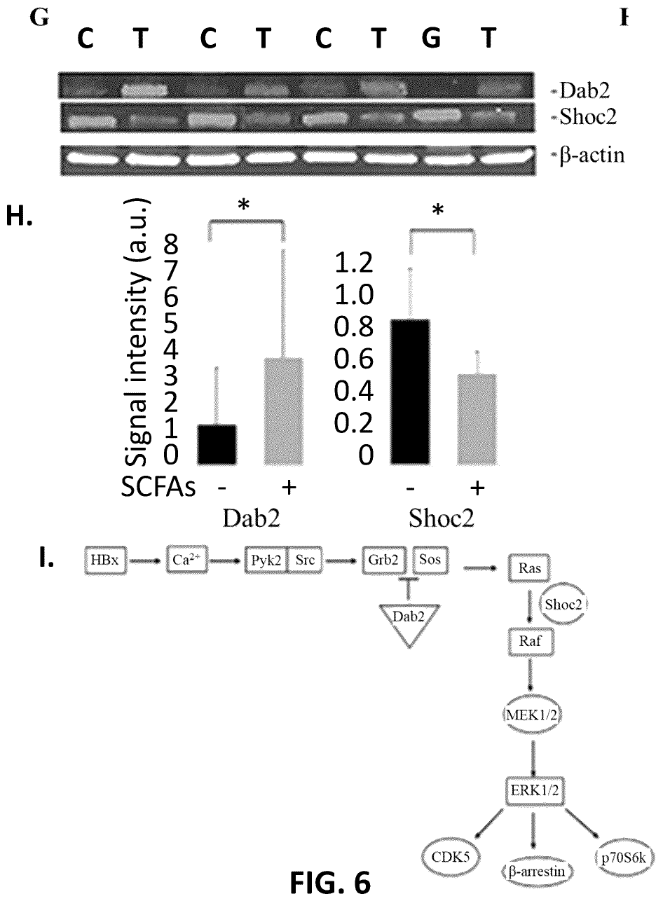

The results above showed that SCFA treatment was associated with a decrease in the size and appearance of HCC nodules in some mice, and smaller tumors in others (FIG. 2, TABLE 3). Since HBx is known to activate Ras in HCC pathogenesis, and many proteins associated with Ras signaling were altered by SCFA treatment (FIG. 6) in 12-month old HBxTg mouse livers, this pathway was chosen for further analysis. Proteomics data revealed that several proteins directly associated with Ras were decreased by SCFA treatment, such as the upstream Ras-Raf scaffold protein Shoc2, and downstream activator, MEK2. Proteins downstream of Ras were also decreased including CDK5 and p70S6k. The tumor suppressor and Ras inhibitor, Dab2, was increased upon treatment (TABLE 4, FIG. 6). Taken together, the results of proteomics suggest that the ability of SCFAs to delay the development of neoplastic lesions involves the downregulation of important cancer-promoting proteins in the Ras pathway.

### Example 4

### Validation of Differentially Expressed RAS Related Proteins

The Ras/Raf/MEK/ERK signaling cascade drives cell proliferation, differentiation, apoptosis, and tumorigenesis, and is activated in 50-100% of human HCCs. HBx activates Ras by promoting Shc-Grb2-Sos complex formation. Dab2 inhibits Ras activation by competitively blocking the formation of this complex. To validate the results of the proteomics with regard to the Ras pathway, IHC was performed. For HBx, many of the SCFA-treated and placebo-treated mice showed diffuse, lobular and scattered tissue staining in the nuclear and cytoplasmic compartments of cells, although the differences were not statistically significant. However, the intensity of HBx staining decreased in SCFA-treated mice compared to controls (compare FIG. 6, Panels A and D, P<0.02). Cytoplasmic staining was observed in liver samples evaluated from all mice, while half the animals also had nuclear HBx. When consecutive tissue slides from these same mice were stained for Dab2, scattered single cells showed weak cytoplasmic and sometimes nuclear staining for Dab2 in both SCFA-treated and placebo-treated mice, but these differences were not statistically significant. However, Dab2 staining was more widespread in a larger number of scattered cells and lobular tissue staining was observed in SCFA-treated compared to control tissues (compare FIG. 6, Panels B and E, P<0.025). Staining specificity was demonstrated using normal rabbit IgG (FIG. 6, Panel C) or an irrelevant monoclonal antibody (FIG. 6, Panel F). Western blotting showed that Dab2 was up-regulated 2.6-fold in SCFA-treated compared to control mice, and that for Shoc2, which is required for the Ras to activate Raf, the inverse was observed (FIG. 6, Panels G and H; P<0.01). Confirmation of Shoc2 and Dab2 alterations detected by proteomics and by western blotting further supports that SCFAs may downregulate the Ras pathway. In addition, the up-regulation of Dab2, which suppresses Ras signaling, should also result in suppressed levels of down-stream effectors, such as MEK1/2, cyclin dependent kinase 5 (CDK5), β-arrestin1, and the ribosomal kinase p70s6k, all of which were highly expressed in the livers of PBS-treated mice but undetectable in mice treated with SCFAs in the proteomics analysis (FIG. 6, Panel I).

FIG. 6 shows immunohistochemical staining for HBx (Panels A and D), Dab2 (Panels B and E), normal IgG (Panel C), or rabbit pre-immune serum (Panel F) in 12-month old mouse livers from animals treated with PBS (Panels A-C) or SCFAs (Panels D-F). Panel G shows a representative Western blot of Dab2 and Shoc2 from treated (T) compared to control (C) livers. Panel H shows a summary of differentially expressed Dab2 and Shoc2 from treated (n=12) compared to control (n=12) mice (*P<0.01). Panel I shows a summary of proteomics data of SCFAs on Ras-related proteins in 12-month old HBxTg mouse livers. Proteins with increased expression by SCFA treatment are indicated by a triangle, proteins decreased by SCFA treatment are indicated by circles. Proteins in the Ras pathway that were not differentially expressed by SCFA treatment are indicated by a box. Dab2, disabled homolog 2; HBx, hepatitis B x; SV40, simian virus 40; SCFAs, short chain fatty acids.

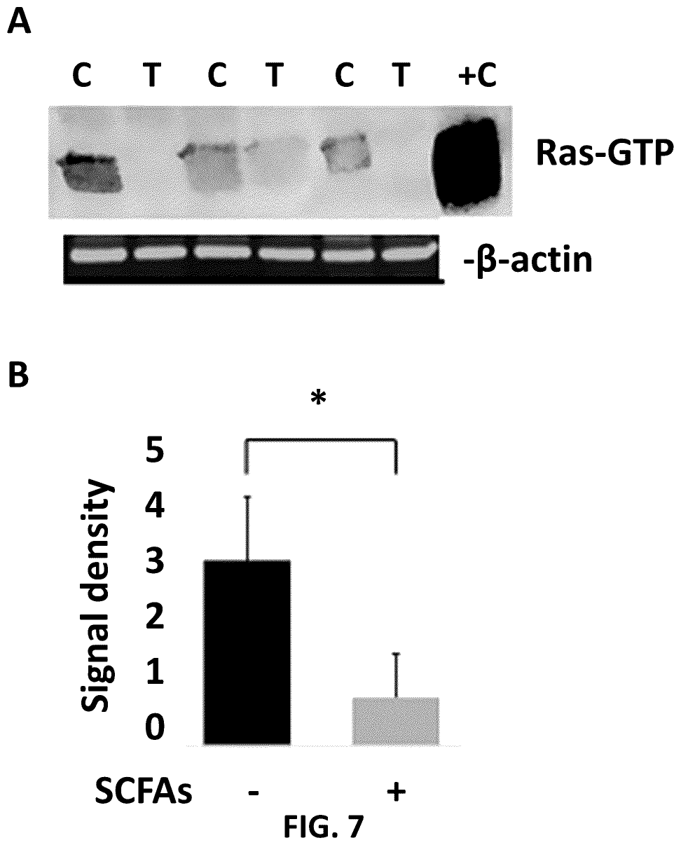

Ras is a small GTPase that cycles between the active, GTP-bound form, and the inactive, GDP-bound form. Experiments were then designed to determine the levels of the active form of Ras (Ras-GTP) in the 12-month old SCFA-fed and PBS-fed livers using a GST-pulldown assay. SCFA-treated HBxTg mouse livers showed a 4-fold decrease in expression of Ras-GTP compared to control samples (FIG. 7), demonstrating that SCFA treatment is associated with decreased levels of active Ras (P<0.001), but no changes in the levels of total Ras protein, as determined by proteomics (TABLE 4) and western blotting (data not shown). Given that Ras stimulates many processes important to carcinogenesis, and is known to be activated in HCC, these results suggest that the ability of SCFAs to inhibit Ras contributes to the delayed pathogenesis of HCC in this animal model.

FIG. 7, Panel A shows a pulldown assay for activated Ras in three different mice treated as controls (C) with PBS and three mice treated with the test compounds (T), SCFAs, prior to analysis. Panel B shows a summary of Ras pulldown in seven mice treated with PBS compared to another seven treated with SCFAs (*P<0.001). Signal density was measured in arbitrary units (a.u.). GTP, guanosine triphosphate; PBS, phosphate buffered saline; SCFAs, short chain fatty acids.

### Example 5

### Discussion

Experiments were designed to identify changes occurring in the liver of HBxTg mice at the time when HCC nodules develop. SCFAs significantly reduced the number of mice that developed dysplasia by 9 months of age (TABLE 3). In 12-month old mice, treatment reduced the number of mice that developed dysplasia and HCC (TABLE 3). Unexpectedly, the incidence of steatosis was higher in both treated groups compared to controls, although these differences were not statistically significant. This may be due to a higher intake of fatty acids in the treated group, as previously reported. The 12-month group treated with SCFAs had smaller tumors than control mice (FIG. 2). This result suggests that SCFA treatment may either delay tumor onset and/or directly affect the growth of established tumors. The latter was confirmed in vitro, in which SCFAs inhibited the growth of the HBx positive human HCC cell lines, Huh7x and Hep3Bx (FIG. 4). These findings extend prior observations showing that butyrate inhibits the proliferation of HepG2.2.15 cells. Butyrate may also delay or prevent tumor progression by promoting differentiation of hepatoma cell lines. Treatment of primary human hepatocytes with SCFAs resulted in no discernable loss of viability (FIG. 4). This result suggests that pathways effecting the viability of tumor cells were much more sensitive to SCFAs than the same pathways in normal hepatocytes.

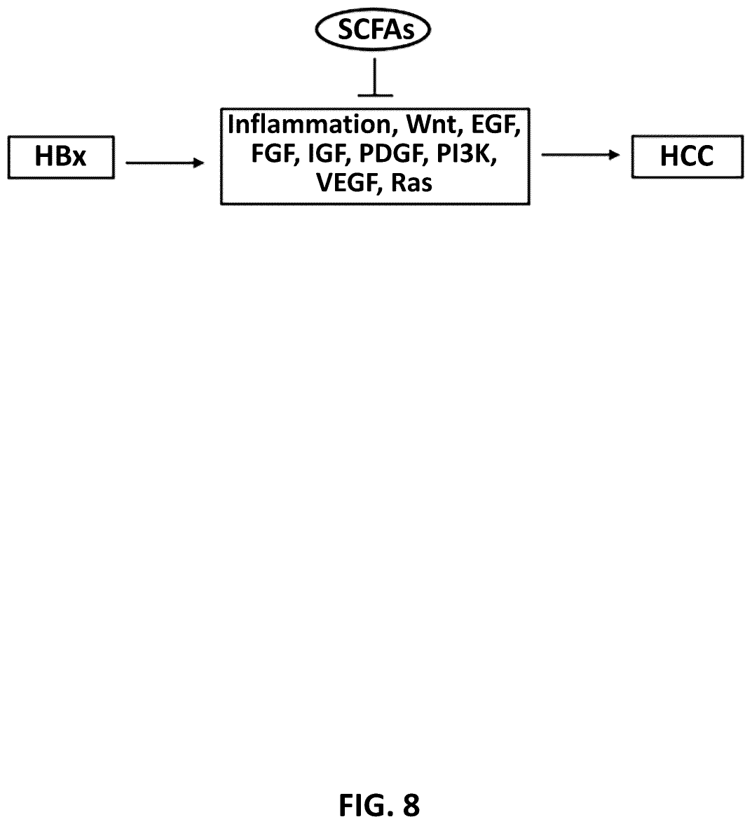

To distinguish the nature of these changes in signaling pathways and patterns of gene expression that underlie the effects of SCFAs, proteomics was conducted in multiple liver samples harvested from 12-month old mice. The results showed differential expression of many proteins in a variety of biological processes (FIG. 5), thereby underscoring the probable pleiotropic effects of SCFAs upon multi-step carcinogenesis. Pathway analysis of these differentially expressed proteins in the liver revealed suppressed Ras, PI3K, VEGF, TGF-β, interferon signaling, and inflammation associated pathways, among others in response to SCFA treatment (TABLE 4). These same pathways are known to be activated by HBx (FIG. 8).

FIG. 8 shows a summary of pathway analysis of 12-month old SCFA-treated vs control HBxTg livers. The pathways shown were consistently up-regulated by HBx and down-regulated by SCFAs. EGF, epidermal growth factor, FGF, fibroblast growth factor; HBx, hepatitis B x; HBxTg, hepatitis B x transgenic; HCC, hepatocellular carcinoma; IGF, insulin-like growth factor; PDGF, platelet derived growth factor; PI3K, phosphoinositide 3-kinase; SCFAs, short chain fatty acids; VEGF, vascular endothelial growth factor.

In addition, NF-κB, which is constitutively activated by HBx and contributes importantly to the pathogenesis of HCC, is epigenetically down-regulated by SCFAs, especially butyrate. Among the downregulated cancer-related pathways herein, Ras, PI3K, VEGF, FGF, and EGF are all activated by HBx in HCC, and all crosstalk with NF-κB, thereby suggesting that NF-κB inhibition may also occur. HBx protects infected cells from apoptosis by promoting activity of the PI3K and Ras pathways. This deregulation was found to promote HCC progression through unrestrained cell proliferation, invasion, and metastasis. Alternations in other pathways involving angiogenesis (VEGF signaling), cell death (apoptosis signaling), immune mediated destruction (T cell activity), sustained proliferative signaling (IGF, Ras, and PDGF), tumor promoting inflammation (chemokine and cytokine signaling), as well as invasion and metastasis (integrin signaling), all of which are down-regulated by SCFAs, are hallmarks of cancer that can underlie the delay in the multiple steps that ultimately result in the development of HCC.

Previously, HBx was shown to synergize with Kras to promote the formation and progression of HCC. This relationship also led to the deregulation of Akt, TGF-β, and β-catenin, among other proteins. Proteomic analysis of HBV-infected tissue confirmed HBx increases oxidative stress through interaction with HIF-1α, a pathway that was also demonstrated to be altered in the proteomic analysis herein. Further analysis of HBV-HCC tissue compared to adjacent non-tumor tissue showed altered expression of β-catenin-related proteins, NF-κB signaling components, ribosomal subunits, ubiquitin-related proteins, respiratory complex and metabolism-related protein, which corroborates changes also detected in the present study. This data presented herein establish that some of the proteins and pathways altered by HBx in the pathogenesis of HCC are reversed by treatment with SCFAs.

Treatment with the multi-kinase inhibitor, sorafenib, and related compounds, has been the standard of care for patients with late HCC. Specifically, the inhibition of the PI3K, PDGF, VEGF, IGF, TGF-β, and Ras pathways by sorafenib is largely regarded as responsible for the drug's chemotherapeutic effect. One problem limiting the efficacy of sorafenib may be that treatment is provided to patients with advanced HCC, and if many of the critical changes leading to cancer already occur prior to the development of tumors, earlier intervention may have prophylactic value. Because SCFAs had no effect on the viability of normal hepatocytes (FIG. 4), SCFAs are specifically toxic to malignant cells, and may provide an alternative approach to limiting toxicity among HBV carriers who already have significant liver damage.

Given that SCFAs reduce the incidence of HCC in 12-month old treated livers, and that the Ras signaling pathway is both stimulated by HBx and is commonly activated in HCC, additional work was carried out to validate that the activity and differential expression of Ras pathway components that were differentially expressed by proteomics. The results in FIG. 6 show increased expression of the tumor suppressor, Dab2, and decreased expression of Shoc2, a downstream Ras signaling pathway component. Ras activity was also significantly diminished by SCFA treatment (FIG. 7). Importantly, epigenetic silencing of Dab2 in HCC has been associated with Ras activation, thereby resulting in HCC initiation and progression. Since Dab2 negatively regulates Ras by competing with Sos binding to Grb2, thereby disrupting the formation of the Sos/Grb2 complex, and suppressing consequent activation of Ras, this may be one mechanism whereby SCFAs delay the pathogenesis of HCC. Activation of this pathway has also been found in several other human cancers including breast, colon, and prostate cancer, which suggests that SCFAs can be beneficial in the treatment of other cancer types.

The epigenetic silencing of Dab2 has been investigated in other human cancers. Treatment with Trichostatin A (TSA), a well-known HDACi, restored Dab2 expression in nasopharyngeal carcinoma, squamous cell carcinoma, and transitional carcinoma cells. This result indicates that HDAC-mediated chromatin modulation can play a role in Dab2 downregulation. SCFAs may be able to recover expression of epigenetically silenced genes in several ways. SCFAs may block HBx itself thus preventing it from mediating its alterations, as butyrate has been shown to block HBx expression by inhibiting the HDAC, SIRT-1. Butyrate has also been shown to inhibit the expression of HBx in HepG2.2.15 cells, which has also been observed in vivo. At the molecular level, SCFAs may render HDACs ineffective by competitively binding to their active sites, thus preventing the silencing of tumor suppressors. Decreased HBx expression in the liver of SCFA-treated mice (FIG. 6) may also restore the activity of the tumor suppressor p53, which is also known to be inhibited by HBx. In addition, Dab2 was identified as a target of miR-106b in cervical cancer. Dab2 suppression in HCC may be due to the increased expression of miR-106b, which is promoted by HBx. Butyrate decreased the expression of miR-106b which was accompanied by decreased cell proliferation. Thus, the targeting of HBx functions through use of SCFAs can provide a novel, nontoxic approach for the treatment of HBV carriers with CLD who are at high risk for the development of cirrhosis and HCC.

### Example 6

### Materials and Methods

A. Short chain fatty acids (SCFAs): SCFAs were purchased and used without further purification. The SCFAs were sodium salts of butyrate, propionate, and acetate.

B. Mice and treatments: In HBxTg mice (C57B1/6×DBA), hepatitis and steatosis develops in animals by 6 months of age, dysplasia by 9 months, and HCC nodules by 12 months. Sibling littermates, consisting of 6-month and 9-month old mice of both genders, were treated five days per week during daylight hours with SCFAs or phosphate buffered saline (PBS) by oral gavage for three months. Concentrations of each SCFA were 40 mM of butyrate, 67.5 mM of acetate, and 25.9 mM of propionate. After three months of treatment, livers were removed and then examined histologically. Tumor diameters were measured using a caliper for small (<0.5 cm), medium (0.5-1.0 cm), and large (>1 cm) nodules. Liver samples from three mice in the 12-month old group were prepared for proteomics (see below).

Controls were sibling littermates. All mice were fed the same diet and given water ad libitum. Mice were not fasted before treatment. Treatment was delivered during the light cycle at approximately the same time each day. Mice were housed in autoclaved cages with Bed-o'cob® and domes for enrichment. After three months of treatment, mice were anesthetized with a ketamine/xylazine cocktail and then perfused with PBS.

FIG. 1 shows an outline of experimental steps of the HBx transgenic (HBxTg) mice study. Asterisks (*) indicate the age of mice in months when mice in each group were euthanized. Bars indicate the duration of treatment from 6-9 months (groups 1 and 2) and 9-12 months (groups 3 and 4) for mice treated with SCFAs (groups 1 and 3) or PBS (groups 2 and 4). H & E, hematoxylin and eosin; IHC, immunohistochemistry; PBS, phosphate buffered saline; SCFAs, short chain fatty acids.

C. Immunohistochemistry: Immunohistochemistry (IHC) analyses were performed using paraffin-embedded sections of tissue samples. Primary antibodies were anti-HBx (anti-99), as well as rabbit polyclonal antibodies against Dab2 and Shoc2.

Livers from HBxTg mice were removed, fixed in formalin, and embedded in paraffin. 5-μm thick tissue slices were prepared from these paraffin blocks. For immunohistochemistry (IHC), slides were deparaffinized, dehydrated, incubated for 30 minutes in Unitrieve antigen retrieval solution, heated to 60° C., and stained using the UltraVision® detection system. Normal mouse immunoglobulin G (IgG) was used as a control for anti-Dab2 while pre-bleed rabbit serum from the same animal immunized to produce anti-99 (anti-HBx peptide antibodies) was used as a control for HBx IHC. For Dab2 staining, rabbit polyclonal antibodies were used, and normal rabbit IgG was used as control. Antibody dilutions were used as recommended by the manufacturer. IHC results were recorded as + (<20% positive cells), ++ (20-70% positive cells), and +++ (>70% positive cells). IHC was also evaluated at the cellular level as scattered, (individual cells positive), lobular (groups of positive cells), or diffuse (most cells in a section positive). Subcellular localization for IHC was also assessed as membranous (M), nuclear (N), or cytoplasmic (C). Liver histopathology was evaluated using hematoxylin and eosin staining. Slides were evaluated independently by two investigators.

D. SDS/PAGE and Western Blotting: Protein lysates were prepared from snap frozen liver tissue from 12-month old control and SCFA-treated HBxTg mouse liver samples. Previously snap frozen liver tissues were homogenized in lysis buffer with protease inhibitor cocktail. Cell debris was removed by centrifugation twice at 14,000 g for 15 minutes. Protein extracts from cells were prepared using the same lysis buffer. For western blotting (WB), 100 μg of protein extracts from liver tissues were separated by SDS-polyacrylamide gel electrophoresis and transferred to nitrocellulose membranes. Membranes were incubated with anti-Dab2 or anti-Shoc2, and anti-β-actin. The blots were developed using the Odyssey ® western blotting kit. Secondary antibodies were IRDye® goat anti-mouse for β-actin detection or goat anti-rabbit IgG for Dab2 and Shoc2. Visualization was performed by Odyssey® Fc imaging system and quantification by Image Studio® software.

E. RAS Activity assay: A Ras activation assay was performed on control and SCFA-treated HBxTg mouse liver samples to isolate GTP bound Ras according to manufacturer's instructions. Once isolated, the samples were separated by SDS-PAGE and transferred to nitrocellulose membranes. Membranes were incubated with anti-ras and secondary antibody IRDye® goat anti-rabbit IgG. The blots were developed using the Odyssey® western blotting kit. Blots were visualized using Odyssey® Fc imaging system and quantified by Image Studio® software.

F. Cell culture: Huh7 and Hep3B cells were stably transduced with HBx gene by recombinant retroviruses (referred to as Huh7x and Hep3Bx, respectively) and cultured without the selection of individual clones as previously described. Primary human hepatocytes were purchased from Zen-Bio, Inc. and cultured according to manufacturer's instructions.

G. Cell viability and treatment: Cells were plated in 96 well plates in complete DMEM and incubated in 5% CO₂ overnight. Treatment consisted of different concentrations of SCFAs (0, 1, 5, and 10 mM) for 24 hours. Cell survival was then determined in triplicate using the MTS assay according to manufacturer's instructions.

H. Proteomics and data analysis: Liver tissues from 12-month old control (n=3) and SCFA-treated (n=3) HBxTg mice were homogenized and extracted proteins were digested. Peptides were acidified and loaded onto an activated in-house-made cation stage tip, purified and eluted into three fractions. Mass spectrometry analysis was performed on these fractions. Differentially altered proteins were organized into functional pathways using the PANTHER (protein analysis through evolutionary relationships) classification system.

Samples were centrifuged at 14,000 g for 10 minutes. After protein concentration was determined using the Bradford assay, 100 μg protein was digested with the enzyme trypsin. Samples were fractionated and desalted according to in-StageTip processing protocol.

Label-free proteomic analysis was performed independently on these fractions from each mouse in each group. Peptide mixtures were fractionated by ion exchange chromatography and then identified by Q Exactive® mass spectroscopy using MaxQuant® software and further analyzed by PANTHER. Electrospray ionization (ESI) was delivered with an emitter (ID 30 μM, 40 mm length) at a spray voltage of −1800 V. MS/MS fragmentation was performed on the ten most abundant ions in each spectrum using collision-induced dissociation with dynamic exclusion (excluded for 10.0 s after one spectrum), with automatic switching between MS and MS/MS modes. The peptide false discovery rate (FDR) was 0.01, protein FDR 0.01, minimum peptide length was 7 amino acids, and the minimum razor and unique peptides was: 1, min. The generated peak list was processed through the Andromeda® software and searched against the SwissProt mouse database (release 2018_01; 16,950 sequences). Andromeda search parameters were set as Mus musculus (species); trypsin (enzyme); carbamidomethyl (fixed modification) on Cys; (variable modification), methionine oxidation and acetyl (protein N-term); 7 ppm mass tolerance for precursor peptide ions and 20 ppm mass tolerance for product ions. Data were filtered at 1% protein and peptide spectrum matches (PSMs) FDR.

I. Statistics: All data were analyzed using Excel® or GraphPad® software and further statistical analyses were performed as outlined below.

For proteomics, a t-test was performed on quantified proteins on select proteins with low variance within a group and on proteins that were differentially expressed to statistical significance between groups. Those proteins that were differentially expressed (greater than two-fold difference in magnitude compared with control mice and P<0.05) were selected for PANTHER pathway analysis. Additionally, those proteins that were detected in the majority or all samples from one group and no samples of the comparison group were selected for pathway analysis and literature searches.

Chi square analysis was used to determine significance between the percentage of treated vs. control HBxTg mouse livers that developed dysplasia and HCC. The Student's t test was used to evaluate significance in dysplastic nodule development in 9-month old treated vs control HBxTg mice as well as tumor development in 12-month old treated vs control HBxTg mice. The difference in staining intensity of Dab2 between treated vs controls was evaluated by chi square analysis. The difference in cell viability in cell lines was determined by Student's t test. Statistical significance was considered when P<0.05.

### Example 7

### Treatment of HBx Transgenic Mice with Formulation Comprising Butyrate, Propionate, and Magnesium Hydroxide

HBx transgenic mice (C57B1/6×DBA) are created and modified such that the mice develop hepatitis and steatosis by 6 months of age, dysplasia by 9 months of age, and HCC nodules by 12 months of age. Sibling littermates consisting of 6-month and 9-month old mice of both genders are treated 7 days per week during daylight hours with a Formulation 1 (40 mM butyrate, and 25.9 mM of propionate, and 9 mM Mg(OH)₂) or treated with phosphate buffered saline (PBS). Formulation 1 or PBS are administered by adding Formulation 1 or PBS to the drinking water for three months. Water is changed daily with fresh Formulation 1 or PBS samples. Each group of mice consist of 10 mice with approximately equal numbers of male and female mice.

After three months of treatment with Formulation 1 (group 1) or placebo (group 2) from 6-9 months of age; or three months of treatment with Formulation 1 (group 3) or placebo (group 4) from 9-12 months of age; mice are bled out, euthanized, and livers are removed for further analyses. For additional experiments, mice are treated for 9 months (ages 3-12 months of age) with Formulation 1 (group 5) or PBS (group 6). Blood samples are screened for pro-inflammatory and anti-inflammatory markers and for the frequency of CD8+ T cells and FoxP3+ T cells by flow cytometry. HBx expression is assessed in the livers of Formulation 1 and PBS treated mice.

### Example 8

### Treatment of HBV-Associated Liver Inflammation with Formulation Comprising Butyric Acid, Propionic Acid, and Magnesium Hydroxide

A total of 100 patients with a median age of 60 are recruited. The patients have ongoing HBV infections and associated liver inflammation and/or cirrhosis and no contraindication for butyrate. The patients are dosed with a formulation comprising 1 g sodium butyrate, 100 mg sodium propionate, and 0.0055 mg magnesium hydroxide three times daily or given placebo and included in a screening program for hepatocellular carcinoma (HCC). The subjects are followed for a median of 5 years. Just prior to treatment, patients are screened for ALT/AST, GGT; liver stiffness by fibroscan; pro-inflammatory and anti-inflammatory cytokines in blood (TNFalpha, IFNgamma, IL-2, IL-4, IL5-IL-10); and HBV markers (HBeAg, anti-HBe, and HBV DNA). The screenings are repeated yearly until completion of the study. Patients are analyzed by multivariate analysis for incidence of chronic liver disease (hepatitis and fibrosis), liver cancer, chronic liver disease related death (usually from cirrhosis), or transplantation in the treated and untreated cohorts.

### EMBODIMENTS

The following non-limiting embodiments provide illustrative examples of the invention, but do not limit the scope of the invention.

### Embodiment 1

A method of treating hepatocellular carcinoma, the method comprising administering to a subject in need thereof of a pharmaceutical composition, the pharmaceutical composition comprising a therapeutically-effective amount of a first compound that is a first short chain fatty acid or a pharmaceutically-acceptable salt thereof, a therapeutically-effective amount of a second compound that is a second short chain fatty acid or a pharmaceutically-acceptable salt thereof, and magnesium hydroxide.

### Embodiment 2

The method of embodiment 1, wherein the hepatocellular carcinoma is hepatitis B virus-associated hepatocellular carcinoma.

### Embodiment 3

The method of embodiment 1 or 2, wherein the administering is oral.

### Embodiment 4

The method of embodiment 1 or 2, wherein the administering is subcutaneous.

### Embodiment 5

The method of embodiment 1 or 2, wherein the administering is intravenous.

### Embodiment 6

The method of any one of embodiments 1-5, wherein the first compound is butyric acid.

### Embodiment 7

The method of embodiment 6, wherein the first compound is sodium butyrate.

### Embodiment 8

The method of embodiment 1, wherein the therapeutically-effective amount of the first compound is from about 500 mg to about 4000 mg.

### Embodiment 9

The method of embodiment 1, wherein the therapeutically-effective amount of the first compound is about 2000 mg.

### Embodiment 10

The method of embodiment 1, wherein the therapeutically-effective amount of the first compound is about 3000 mg.

### Embodiment 11

The method of embodiment 1, wherein the therapeutically-effective amount of the first compound is about 4000 mg.

### Embodiment 12

The method of any one of embodiments 1-11, wherein the second compound is propionic acid.

### Embodiment 13

The method of embodiment 12, wherein the second compound is sodium butyrate.

### Embodiment 14

The method of any one of embodiments 1-13, wherein the therapeutically-effective amount of the second compound is from about 50 mg to about 500 mg.

### Embodiment 15

The method of any one of embodiments 1-13, wherein the therapeutically-effective amount of the second compound is about 150 mg.

### Embodiment 16

The method of any one of embodiments 1-13, wherein the therapeutically-effective amount of the second compound is about 250 mg.

### Embodiment 17

The method of any one of embodiments 1-13, wherein the therapeutically-effective amount of the second compound is about 400 mg.

### Embodiment 18

The method of any one of embodiments 1-17, wherein the pharmaceutical composition comprises at most about 0.5% (w/w) of magnesium hydroxide.

### Embodiment 19

The method of any one of embodiments 1-17, wherein the pharmaceutical composition comprises at most about 0.3% (w/w) of magnesium hydroxide.

### Embodiment 20

The method of any one of embodiments 1-17, wherein the pharmaceutical composition comprises at most about 0.1% (w/w) of magnesium hydroxide.

### Embodiment 21

The method of any one of embodiments 1-20, wherein the pharmaceutical composition further comprises a pharmaceutically-acceptable excipient.

### Embodiment 22

The method of embodiment 21, wherein the pharmaceutically-acceptable excipient is a cellulose.

### Embodiment 23

The method of embodiment 21, wherein the pharmaceutically-acceptable excipient is methylcellulose.

### Embodiment 24

The method of embodiment 21, wherein the pharmaceutically-acceptable excipient is hydroxypropyl methylcellulose.

### Embodiment 25

The method of any one of embodiments 1-24, wherein the pharmaceutical composition further comprises an enteric coating.

### Embodiment 26

The method of embodiment 25, wherein the enteric coating is a hydroxypropyl methylcellulose capsule.

### Embodiment 27

The method of any one of embodiments 1-26, wherein the pharmaceutical composition is formulated as a tablet.

### Embodiment 28

The method of any one of embodiments 1-26, wherein the pharmaceutical composition is formulated as a capsule.

### Embodiment 29

The method of any one of embodiments 1-28, wherein the subject is human.

### Embodiment 30

A pharmaceutical composition comprising a therapeutically-effective amount of a first compound that is butyric acid or a pharmaceutically-acceptable salt thereof, a therapeutically-effective amount of a second compound that is propionic acid or a pharmaceutically-acceptable salt thereof, and magnesium hydroxide.

### Embodiment 31

A pharmaceutical composition consisting essentially of a therapeutically-effective amount of a first compound that is butyric acid or a pharmaceutically-acceptable salt thereof, a therapeutically-effective amount of a second compound that is propionic acid or a pharmaceutically-acceptable salt thereof, and magnesium hydroxide.

### Embodiment 32

The pharmaceutical composition of embodiment 30 or 31, wherein therapeutically-effective amount of the first compound is from about 500 mg to about 4000 mg.

### Embodiment 33

The pharmaceutical composition of embodiment 30 or 31, wherein the therapeutically-effective amount of the first compound is about 2000 mg.

### Embodiment 34

The pharmaceutical composition of embodiment 30 or 31, wherein the therapeutically-effective amount of the first compound is about 3000 mg.

### Embodiment 35

The pharmaceutical composition of embodiment 30 or 31, wherein the therapeutically-effective amount of the first compound is about 4000 mg.

### Embodiment 36

The pharmaceutical composition of embodiment 30 or 31, wherein the pharmaceutically-acceptable salt of the first compound is sodium butyrate.

### Embodiment 37

The pharmaceutical composition of any one of embodiments 30-36, wherein the therapeutically-effective amount of the second compound is from about 50 mg to about 500 mg.

### Embodiment 38

The pharmaceutical composition of any one of embodiments 30-36, wherein the therapeutically-effective amount of the second compound is about 150 mg.

### Embodiment 39

The pharmaceutical composition of any one of embodiments 30-36, wherein the therapeutically-effective amount of the second compound is about 250 mg.

### Embodiment 40

The pharmaceutical composition of any one of embodiments 30-36, wherein the therapeutically-effective amount of the second compound is about 400 mg.

### Embodiment 41

The pharmaceutical composition of any one of embodiments 30-40, wherein the pharmaceutically-acceptable salt of the second compound is sodium propionate.

### Embodiment 42

The pharmaceutical composition of any one of embodiments 30-41, wherein the pharmaceutical composition comprises at most about 0.5% (w/w) of magnesium hydroxide.

### Embodiment 43

The pharmaceutical composition of any one of embodiments 30-41, wherein the pharmaceutical composition comprises at most about 0.3% (w/w) of magnesium hydroxide.

### Embodiment 44

The pharmaceutical composition of any one of embodiments 30-41, wherein the pharmaceutical composition comprises at most about 0.1% (w/w) of magnesium hydroxide.

### Embodiment 45

The pharmaceutical composition of any one of embodiments 30-44, further comprising a pharmaceutically-acceptable excipient.

### Embodiment 46

The pharmaceutical composition of embodiment 45, wherein the pharmaceutically-acceptable excipient is a cellulose.

### Embodiment 47

The pharmaceutical composition of embodiment 45, wherein the pharmaceutically-acceptable excipient is methylcellulose.

### Embodiment 48

The pharmaceutical composition of embodiment 45, wherein the pharmaceutically-acceptable excipient is hydroxypropyl methylcellulose.

### Embodiment 49

The pharmaceutical composition of any one of embodiments 30-48, wherein the pharmaceutical composition further comprises an enteric coating.

### Embodiment 50

The pharmaceutical composition of embodiment 49, wherein the enteric coating is a hydroxypropyl methylcellulose capsule.

### Embodiment 51

The pharmaceutical composition of any one of embodiments 30-50, wherein the pharmaceutical composition is formulated as a tablet.

### Embodiment 52

The pharmaceutical composition of any one of embodiments 30-50, wherein the pharmaceutical composition is formulated as a capsule.

What is claimed is:

1. A method of treating hepatocellular carcinoma, the method comprising administering to a subject in need thereof of a pharmaceutical composition, the pharmaceutical composition comprising a therapeutically-effective amount of a first compound that is a first short chain fatty acid or a pharmaceutically-acceptable salt thereof, a therapeutically-effective amount of a second compound that is a second short chain fatty acid or a pharmaceutically-acceptable salt thereof, and magnesium hydroxide.

2. The method of claim 1, wherein the hepatocellular carcinoma is hepatitis B virus-associated hepatocellular carcinoma.

3. The method of claim 1 or 2, wherein the administering is oral.

4. The method of claim 1 or 2, wherein the administering is subcutaneous.

5. The method of claim 1 or 2, wherein the administering is intravenous.

6. The method of claim 1, wherein the first compound is butyric acid.

7. The method of claim 6, wherein the first compound is sodium butyrate.

8. The method of claim 1, wherein the therapeutically-effective amount of the first compound is from about 500 mg to about 4000 mg.

9. The method of claim 1, wherein the therapeutically-effective amount of the first compound is about 2000 mg.

10. The method of claim 1, wherein the therapeutically-effective amount of the first compound is about 3000 mg.

11. The method of claim 1, wherein the therapeutically-effective amount of the first compound is about 4000 mg.

12. The method of claim 1, wherein the second compound is propionic acid.

13. The method of claim 12, wherein the second compound is sodium butyrate.

14. The method of claim 1, wherein the therapeutically-effective amount of the second compound is from about 50 mg to about 500 mg.

15. The method of claim 1, wherein the therapeutically-effective amount of the second compound is about 150 mg.

16. The method of claim 1, wherein the therapeutically-effective amount of the second compound is about 250 mg.

17. The method of claim 1, wherein the therapeutically-effective amount of the second compound is about 400 mg.

18. The method of claim 1, wherein the pharmaceutical composition comprises at most about 0.5% (w/w) of magnesium hydroxide.

19. The method of claim 1, wherein the pharmaceutical composition comprises at most about 0.3% (w/w) of magnesium hydroxide.

20. The method of claim 1, wherein the pharmaceutical composition comprises at most about 0.1% (w/w) of magnesium hydroxide.

21. The method of claim 1, wherein the pharmaceutical composition further comprises a pharmaceutically-acceptable excipient.

22. The method of claim 21, wherein the pharmaceutically-acceptable excipient is a cellulose.

23. The method of claim 21, wherein the pharmaceutically-acceptable excipient is methylcellulose.

24. The method of claim 21, wherein the pharmaceutically-acceptable excipient is hydroxypropyl methylcellulose.

25. The method of claim 1, wherein the pharmaceutical composition further comprises an enteric coating.

26. The method of claim 25, wherein the enteric coating is a hydroxypropyl methylcellulose capsule.

27. The method of claim 1, wherein the pharmaceutical composition is formulated as a tablet.

28. The method of claim 1, wherein the pharmaceutical composition is formulated as a capsule.

29. The method of claim 1, wherein the subject is human.
